(19) United States
(12) Patent Application Publication
(10) Pub. No.: US 2025/0072835 A1
(43) Pub. Date: Mar. 6, 2025

(54) INTRAORAL DEVICES FOR MONITORING USE OF DENTAL APPLIANCES

(71) Applicant: Align Technology, Inc., San Jose, CA (US)

(72) Inventors: Byong-Ho PARK, San Jose, CA (US); Zijie ZHU, San Jose, CA (US); Fred TING, San Jose, CA (US); Wesly WONG, Cupertino, CA (US); Zhichao ZHANG, Walnut Creek, CA (US)

(21) Appl. No.: 18/820,196

(22) Filed: Aug. 29, 2024

Related U.S. Application Data
(60) Provisional application No. 63/579,502, filed on Aug. 29, 2023.

(51) Int. Cl. A61B 5/00; A61B 5/01; A61C 7/08

(52) U.S. Cl. CPC A61B 5/6843; A61B 5/01; A61C 7/08; A61B 2560/0252; A61B 2562/0247; A61B 2562/0252; A61B 2562/0271

(57) ABSTRACT

Apparatuses and methods for monitoring wear status of a dental appliance may include a sensing module that sensing the temperature and one or more additional modalities (e.g., capacitance, impedance, pressure force) around a dental appliance to track when the user is wearing or not wearing the dental appliance. These sensing modules may also determine compliance more accurately with a dental treatment plan.

### CLAIM OF PRIORITY

This patent application claims priority to U.S. Provisional Patent Application No. 63/579,502, titled “INTRAORAL APPARATUSES CONFIGURED TO MONITORING USE,” and filed on Aug. 29, 2023, herein incorporated by reference in its entirety.

### BACKGROUND

During orthodontic treatment, the dental practitioner may rely on the patient to comply with the prescribed dental appliance usage. In some instances, a patient may not wear a dental appliance as prescribed by the dental practitioner. Failure to wear a dental appliance correctly or failure to wear the dental appliance for periods of time extending beyond what is recommended may interrupt a dental treatment plan and lengthen the overall period of treatment. There is a need for methods and apparatuses that allow monitoring of the wearing and/or effects of intraoral appliances. Described herein are methods and apparatuses for performing such monitoring.

In addition to monitoring patient wearing behavior, there are many other behavioral states of the user that it would be beneficial to monitor, including but not limited to, cleaning of the dental appliance, eating/drinking while wearing the dental appliance, and/or general patient health states, such as body temperature and respiration. The methods and apparatuses described herein may also be used to beneficially monitor these patient behavioral states.

### SUMMARY OF THE DISCLOSURE

Described herein are methods and apparatuses for monitoring a dental appliance (e.g., an aligner, a retainer, a mouth guard, a palatal expander) and events or use states of the device. This monitoring may involve continuous or semi-continuous monitoring of directly measured temperature on or around a dental appliance using a temperature sensor coupled/mounted to the dental appliance) and determining wear-status state (i.e., whether the user is wearing or not wearing the dental appliance). The monitoring can be used to determine and track compliance to a desired plan (e.g., a treatment plan that requires wearing of a dental appliance for a specified number of hours per day and/or wearing of the dental appliance during particular time periods), or simply to track usage (i.e., with or without a plan). The methods and apparatuses described herein may also determine ambient temperature, and may use the determined ambient temperature in addition to directly measured temperature. In some cases, the directly measured temperature alone may not provide sufficient information to make a determination. For example, a directly measure temperature that is within a body temperature range may suggest that the dental appliance is being worn, but this would be erroneous if the ambient temperature is also within the body temperature range. As such, it would be advantageous to also know the ambient temperature. As used herein, “ambient temperature” may refer to the temperature outside of the user's (e.g., patient's) body, and in particular outside of the user's mouth in the case of a dental appliance. However, ambient temperature is difficult to continuously and reliably measure using a dental appliance that is not going to always be in an ambient environment (e.g., during its use when worn by a patient, when it is being cleaned, etc.). The methods and systems described herein may present a way to determine ambient temperature based on a plurality of directly measured temperature readings. The ambient temperature can then be used in conjunction with the directly measured readings to determine, for example, the wear-status state, as described herein. In some examples, a second corroborating sensor (e.g., a capacitive sensor, a voltage sensor, a piezoelectric sensor, an optical sensor) that measures a parameter correlated to proximity with a body tissue (e.g., tissues in the intraoral cavity) may be used to provide further accuracy in, for example, determining wear-status state. Although this disclosure focuses on wear-status state, it contemplates determining particular activities/events (e.g., drinking/eating, breathing rate, exercise, cleaning the dental appliance), determining one or more health indicators (e.g., changes in body temperature, including in some examples core body temperature), etc. Although the disclosure focuses on dental appliances, the disclosure contemplates using the described methods and systems for any suitable appliance for which compliance status needs to be monitored. For example, the system can be used for any other wearable device (e.g., a glucose sensor, an optical sensor, a blood oxygenation level sensor, a respiration sensor).

In general, these method and apparatuses may track the ambient temperature and/or the one or more behavioral states over a period of time, including continuously and/or for a discrete period of time. For example, in some cases these methods and apparatuses may be used continuously after being turned on. In some examples the methods and/or apparatuses may be configured to track ambient temperature and/or one or more behavioral states for a predetermined period of time (e.g., minutes, hours, days, weeks, etc.) after being turned on. The methods and apparatuses described herein may periodically (e.g., discretely) detect temperature and/or one or more behavioral states and may update continuously or periodically (at an adjustable or determined frequency, e.g., every 0.01 seconds, every 0.05 seconds, every 0.1 second, every 0.5 second, every 1 second, every 1.5 seconds, every 2 seconds, every 2.5 seconds, every 3 seconds, every 4 seconds, every 5 seconds, every 7.5 seconds, every 10 seconds, every 15 seconds, every thirty seconds, every minute, every 2 minutes, every 2.5 minutes, every 5 minutes, etc.). These methods and apparatuses may sense temperature and/or another parameter such as an electrical property (e.g., capacitance, resistance, impedance, etc.) using one or more temperature sensor and one or more corroborating sensors (e.g., an electrical sensor such as a capacitance sensor, an optical sensor, etc.), providing corroboration data. Sensed temperature may be referred to as temperature data and sensed corroboration data, e.g., electrical data (e.g., capacitance data, resistance, etc.) may be referred to as sensed corroboration data such as sensed electrical data (e.g., sensed capacitance data, etc.). In some examples, the sensed temperature data and/or sensed corroboration data may be stored, modified (e.g., amplified, filtered, combined, averaged, etc.) and/or may be transmitted to a separate, e.g., remote, processor for processing, including storage, analysis, output and/or further processing.

For example, described herein are methods and apparatuses for deriving and tracking ambient temperature. As will be described in further detail, the disclosed methods and apparatuses enable a dental appliance system that is otherwise “unaware” of its location (e.g., unaware whether it is actually in an ambient environment or inside a user's mouth) to nonetheless determine an ambient temperature (i.e., the temperature outside the user's mouth). This may be done based on sensor data from one or more sensors (one or more of, e.g., a temperature sensor, a capacitance sensor) attached to and/or integrated with a dental appliance of the dental appliance system. The measurement and analysis methods described herein may allow the dental appliance system to not only determine the ambient temperature, but to also determine a wear-status state of the user (e.g., in some cases, based on that determined ambient temperature), and this may be done over an extended period of time to provide continuous, semi-continuous, periodic, or aperiodic monitoring of compliance by the user with a treatment plan. A user may be equivalently referred to herein as a patient or a subject.

These methods and apparatuses described herein may also take corroboration data, such as electrical (e.g., capacitance, resistance, impedance, etc.) readings from one or more electrical sensor(s) on the dental appliance. In some examples the electrical sensor is a capacitance sensor, such as one or more (e.g., a pair of) electrodes. The capacitance senor may be separate from or integrated with the temperature sensor(s). The methods and apparatuses described herein may be configured to read from both the temperature sensor(s) and the electrical sensor(s) concurrently (e.g., at the same time or at approximately the same time), or sequentially. The temperature and corroboration data may be paired and time-stamped. In some examples the apparatus is configured so that the corroboration data is collected only some of the time, e.g., paired or associated with) a subset of the temperature readings. Temperature readings may be taken more frequently. In some examples, temperature readings may be taken less frequently than corroboration data readings.

In general, the methods and apparatuses described herein may examine changes in the temperature and/or corroboration data readings. Thus, in some examples the method or apparatus may look for changes between readings or groups or readings for temperature and/or corroboration data (e.g., capacitance), by comparing the change (from a current reading to a prior reading, or a change in one or more current readings and one or more immediately prior readings) in temperature readings and/or the change in corroboration data (e.g., capacitance) readings to determine if the change from a prior reading (or sequential readings) and a new reading (or group of sequential new readings) is above a threshold value. Relatively large changes may indicate a change reflecting the application and/or removal of the dental appliance.

For example, described herein are apparatuses that are configured to deduce the ambient temperature. As mentioned above and described in greater detail herein, the ambient temperature may be used to determine one or more behavioral states of the user, including (but not limited to) compliance or wearing/not wearing the dental appliance.

Any of these apparatuses may include: a dental appliance having a dental appliance body forming one or more tooth-receiving cavities configured to be worn on a subject's teeth; and a sensing module coupled to the dental appliance body, the sensing module comprising: one or more temperature sensors; one or more processors; and a memory coupled to the one or more processors, the memory storing instructions, that, when executed by the one or more processors, perform a method comprising: setting an ambient temperature value from the one or more temperature sensors during a device initialization stage; receiving one or more new temperature readings from one or more of the temperature sensors; updating the ambient temperature value by comparing the one or more new temperature readings to a body temperature range and comparing a change in the temperature readings to a temperature event threshold amount; and outputting the ambient temperature value.

For example, any of these apparatuses may be configured to deduce and track ambient temperature and may include: a dental appliance having a dental appliance body forming one or more tooth-receiving cavities configured to be worn on a subject's teeth; and a sensing module coupled to the dental appliance body, the sensing module comprising: one or more temperature sensors; one or more proximity sensors (e.g., capacitance sensors); one or more processors; and a memory coupled to the one or more processors, the memory storing instructions, that, when executed by the one or more processors, perform a method comprising: setting an ambient temperature value during a device initialization stage based on one or more initial temperature readings from the one or more temperature sensor; updating the ambient temperature value by taking a new temperature reading from the one or more temperature sensor and setting the ambient temperature value to the new temperature reading if both the ambient temperature value and the new temperature reading are outside of a body temperature range, or setting the ambient temperature value to the new temperature reading if the ambient temperature value is within the body temperature range and the new temperature reading changes more than a temperature event threshold amount from one or more immediately prior temperature readings or if the new temperature reading does not change more than the temperature event threshold amount but a change in a proximity measurement from the one or more proximity sensor exceeds a proximity event threshold amount, otherwise leaving the ambient temperature value the same if the ambient temperature value is within the body temperature range, the new temperature does not change more than the temperature event threshold amount, and the change in the proximity measurement does not exceed the proximity event threshold amount; and outputting the ambient temperature value. The proximity threshold amount may be, e.g., a capacitance threshold amount.

In general, an event threshold, such as a temperature event threshold, a proximity threshold amount, a capacitance event threshold, etc., is a threshold value that indicates that, when the change in a measured or derived value (e.g., temperature change, capacitance change, etc.) has occurred beyond this threshold value, it is likely that event has occurred, such as removing or applying the dental appliance. For example, if there is a change in a parameter beyond the event threshold, it is determined there has been a change in wear-status state. The event thresholds described herein may be preset or predetermined, and/or may be set or adjusted before and/or during use. In some cases the event threshold may be adjusted to adjust the sensitivity of the apparatus or methods described herein.

The sensing module may comprise one or more capacitive sensors. In any of these examples, updating may comprise comparing a change in a proximity measurement from the one or more proximity sensors to a proximity activity threshold amount when the temperature reading is within the body temperature range and the temperature reading changes more than the temperature activity threshold amount. For example, the proximity sensor comprises one or more of: a capacitance sensor or an optical sensor.

In any of these apparatuses the ambient temperature may be updated in an ongoing manner by taking new temperature readings and comparing the new temperature readings and the ambient temperature value (e.g., the current, not-yet-updated ambient temperature value) to a body temperature range, and/or by comparing the new temperature reading to a temperature event threshold amount, and/or by comparing a corroboration data (e.g., capacitance) reading to a corroboration data event threshold amount (e.g., an electrical event threshold amount, such as a capacitance event threshold amount).

For example, updating the ambient temperature value may comprise setting the ambient temperature value to the new temperature reading if both the ambient temperature value and the new temperature reading are outside of the body temperature range, or setting the ambient temperature value to the new temperature reading if the ambient temperature value is within the body temperature range and the new temperature reading changes more than a temperature event threshold amount from one or more immediately prior temperature readings or if the new temperature reading does not change more than the temperature event threshold amount but a change in the capacitance measurement exceeds the capacitance event threshold amount, otherwise leaving the ambient temperature value the same.

As used herein, a dental appliance may be any appropriate appliance, including an aligner, retainer, palatal expander, etc. Thus the dental appliance body may be configured to be worn on and/or over the user's teeth, and may therefore include one or more tooth-receiving cavities configured to be worn on a subject's teeth. The dental appliance may be configured for use with one or more attachments. The sensing module may be configured as an electronic compliance indicator (ECI) apparatus, and may be permanently attached (e.g., bonded to) and/or integrally formed with the dental appliance. In some examples the sensing module may be configured to be inserted into a pocket or receiving region of a dental appliance. In some examples the sensing module may be removably attached to the dental appliance. The sensing module may include a housing enclosing or at least partially enclosing one or more temperature sensors, one or more corroborating sensors (e.g., capacitance sensor, resistive sensor, inductive sensor, etc.) and circuitry, including control circuitry, which may include one or more processors, memory, a clock circuit, communications circuitry (e.g., wireless communication circuitry such as, but not limited to near field communication (NFC), including NFC-to-NFC communication, Wi-Fi, radio (RF, UHF, etc.), infrared (IR), microwave, Bluetooth (including Bluetooth low energy or BLE), magnetic field induction (including NFC), Wimax, Zigbee, ultrasound, etc.), power circuitry and/or a power storage and/or source (e.g., battery, capacitor, etc.). The sensing module may include one or more manual inputs (on/off, etc.) and/or may be controlled wirelessly.

The body temperature range may be, e.g., between about 30 and about 40 degrees Celsius (e.g., between about 32 and about 38 degrees C., between about 33 and 38 degrees C., etc.). The body temperature range may be preprogrammed and/or may be user-set or adjusted. The temperature event threshold amount may be predetermined (e.g., preset), user-set and/or adjustable. For example, the temperature event threshold amount may be determined as a change in the temperature (e.g., 0.5 degrees C., 1 degree C., 1.5 degrees C., 2 degrees C., 2.5 degrees C., 3 degrees C., 3.5 degrees C., 4 degrees C., 4.5 degrees C., 5 degrees C., etc.) or as a change in temperature over time (e.g. per second or portion of a second, such as 1 degree C. per second, 2degrees C. per second, 2.5 degrees C. per second, 3 degrees C. per minute, 3.5 degrees C. per minute, 4 degrees C. per minute, 4.5 degrees C. per minute, 5 degrees C. per minute, etc.). In some examples the corroboration data event threshold amount is a capacitance event threshold amount and may be determined as the change in capacitance (e.g., 1 fF, 2 fF, 3 fF, 4 IF, 5 fF, 7.5 fF, 10 fF, 20 fF, 30 fF, 50 fF, 100 fF, 0.5 pF, 1 pF, etc.). The capacitance event threshold amount may be determined based on the geometry of the capacitance sensor, including the size and/or material forming the capacitive sensor.

For example, any of these apparatuses may be an apparatus for determining ambient temperature from the dental appliance and may include: a dental appliance body forming one or more tooth-receiving cavities configured to be worn on a subject's teeth; and a sensing module coupled to the dental appliance body, the sensing module comprising: one or more temperature sensors; one or more capacitance sensors; one or more processors; and a memory coupled to the one or more processors, the memory storing instructions, that, when executed by the one or more processors, perform a method comprising: setting, during a device initialization stage, an ambient temperature value from the temperature sensor and setting a wear-status state for the dental appliance, wherein the wear-status state is either ‘worn’ or ‘not worn’; updating, on an ongoing basis, the ambient temperature value and the wear-status state using the temperature sensor by comparing one or more new temperature readings from the temperature sensor to a body temperature range and to a temperature event threshold amount, and by comparing a capacitance measurement from the capacitance sensor to a capacitance event threshold amount when the temperature reading is within the body temperature range and the temperature reading changes more than the temperature event threshold amount; and outputting the ambient temperature value.

As mentioned, any of these apparatuses may be apparatuses for determining compliance. Compliance may be indicated by a wear-status state that may indicate either ‘worn,’ meaning the dental appliance is being worn on the user's teeth, or as ‘not worn,’ meaning the dental appliance is not being worn on the user's teeth. The wear-status state may therefore be a Boolean variable or register. In any of these methods and apparatuses the wear-status state may be recorded over time, along with or independently of the derived ambient temperature and/or the determined or sensed temperature from the one or more temperature sensors and/or the determined or sensed corroboration data property (e.g., capacitance).

In practice, the ambient temperature value may be stored in a register or memory and may be accessed by the one or more processors and adjusted as described herein. The values of the ambient temperature value may be stored and appropriately time stamped for later output, as described herein.

For example, an apparatus may include: a dental appliance body forming one or more tooth-receiving cavities configured to be worn on a subject's teeth; and a sensing module coupled to the dental appliance body, the sensing module comprising: one or more temperature sensors; one or more capacitance sensors; one or more processors; and a memory coupled to the one or more processors, the memory storing instructions, that, when executed by the one or more processors, perform a method comprising: setting an ambient temperature value during a device initialization stage based on one or more initial temperature readings from a temperature sensor of a dental appliance and setting a wear-status state for the dental appliance, wherein the wear-status state is either ‘worn’ or ‘not worn’; updating the ambient temperature value by recording a new temperature reading from the temperature sensor and setting the ambient temperature value to the new temperature reading if both the ambient temperature value and the new temperature reading are outside of a body temperature range and setting the wear-status state to ‘not worn’, or setting the ambient temperature value to the new temperature reading and switching the state of the wear-status state, if the ambient temperature value is within the body temperature range and the new temperature reading changes more than a temperature event threshold amount from one or more immediately prior temperature readings or if the new temperature reading does not change more than the temperature event threshold amount but a change in a capacitance measurement from a capacitance sensor on the dental appliance exceeds a capacitance event threshold amount, otherwise leaving the ambient temperature value and the state of the wear-status state the same if the ambient temperature value is within the body temperature range, the new temperature does not change more than the temperature event threshold amount, and the change in the capacitance measurement does not exceed the capacitance event threshold amount; and outputting the ambient temperature value.

In any of these apparatuses and methods updating the ambient temperature value and the wear-status state may include setting the ambient temperature value to a temperature based on the new temperature readings if both the ambient temperature value and the new temperature reading are outside of a body temperature range and setting the wear-status state to ‘not worn’, or setting the ambient temperature value to a temperature based on the new temperature reading and switching the state of the wear-status state if the ambient temperature value is within the body temperature range and the new temperature reading changes more than a temperature event threshold amount from one or more immediately prior temperature readings or if the new temperature reading does not change more than the temperature event threshold amount but a change in a capacitance measurement from a capacitance sensor on the dental appliance exceeds a capacitance event threshold amount, otherwise leaving the ambient temperature value and the state of the wear-status state the same if the ambient temperature value is within the body temperature range, the new temperature does not change more than the temperature event threshold amount, and the change in the capacitance measurement does not exceed the capacitance event threshold amount.

The methods and apparatuses described herein may include a device initialization stage in which the variables that are derived and/or determined such as the ambient temperature value, the wear-status state (e.g., the wear-status state) and/or the body temperature range, and/or the temperature event threshold amount and/or the corroboration data (e.g., capacitance) event threshold amount(s) may be initialized or set. The body temperature range may be read from a memory or buffer, and/or may be input. Similarly the temperature and/or corroboration data event threshold amounts may be read from a memory and/or may be input. The initial values for the ambient temperature value and/or the wear-status state(s) may be derived immediately or shortly after turning the apparatus on.

For example, the apparatus may be configured so that the instructions stored in the memory cause the one or more processors to set the ambient temperature value during the device initialization step by setting the ambient temperature value based on all or some of the one or more initial temperature readings when at least some of the one or more initial temperature readings are outside of the body temperature range. In any of these examples the instructions stored in the memory cause the one or more processors to set the ambient temperature value during the device initialization step by setting the ambient temperature value based on all of the one or more initial temperature readings when all of the one or more initial temperature readings are outside of the body temperature range. In any of these examples the instructions stored in the memory cause the one or more processors to set the ambient temperature value during the device initialization step by setting the ambient temperature value based on the one or more initial temperature readings that are outside of the body temperature range when at least one of the initial temperature readings are within the body temperature range. In any of these apparatuses the instructions stored in the memory may cause the one or more processors to set the ambient temperature value during the device initialization step by taking additional temperature readings from the temperature sensor, and setting the ambient temperature value based on the one or more initial temperature readings when all of the one or more initial temperature readings are within the body temperature range and either a change in the additional temperature readings exceeds the temperature event threshold amount or a change in the capacitance measurement from the capacitance sensor on the dental appliance exceeds the capacitance event threshold amount, alternatively setting the ambient temperature value based on the one or more initial temperature readings and the additional temperature readings after a predetermined device initialization time, when all of the one or more initial temperature readings are within the body temperature range and when neither the change in the additional temperature readings has exceeded the temperature event threshold amount nor the change in the capacitance measurement has exceeded the capacitance event threshold amount.

In general, updating the ambient temperature value may include updating for a predetermined window of time. The window of time may be any appropriate size (e.g., seconds, minutes, hours, days, etc.). In some examples the apparatus may be configured to update the ambient temperature (and/or track the temperature, ambient temperature and one or more wear-status states) until the apparatus receives a stop command or is otherwise turned off. In some examples the window of time is a minimum time (e.g., 5 minutes or more, 10 minutes or more, 15 minutes or more, 20 minutes or more, 25 minutes or more, 30 minute or more, 1 hour or more, 1.5 hours or more, 2 hours or more, 3 hours or more, 5 hours or more, 6 hours or more, 8 hours or more, etc.) or a maximum time (e.g., 5 minutes or less, 10 minutes or less, 15 minutes or less, 20 minutes or less, 25 minutes or less, 30 minute or less, 1 hour or less, 1.5 hours or less, 2 hours or less, 3 hours or less, 5 hours or less, 6 hours or less, 8 hours or less, etc.). In some examples the method or apparatus is configured so that the window of time is open, e.g., so that the tracking of the ambient temperature and/or the wear-status state may be tracked in a continuous manner until stopped, e.g., by turning the tracking off. Thus, any of these apparatuses may include tracking the updates to the ambient temperature value over a predetermined window of time or in a continuous manner.

In general, the apparatuses and method described herein may be configured to output the ambient temperature value (and/or one or more wear-status states) as a stream of time-stamped data. The stream of time-stamped data may also include the temperature and/or corroboration (e.g., capacitance) data may be included with the time-stamped data. The output may be stored and/or transmitted, including wirelessly transmitted to one or more remote processors (e.g., phone, laptop/tablet, cloud, etc.).

In any of these methods and apparatuses, setting may comprise setting the ambient temperature value to the new temperature reading if both the ambient temperature value and the new temperature reading are outside of the body temperature range. For example, setting may comprise setting the ambient temperature value to the new temperature reading if the ambient temperature value is within the body temperature range and the new temperature reading changes more than a temperature event threshold amount from one or more immediately prior temperature readings or if the new temperature reading does not change more than the temperature event threshold amount but a change in the proximity measurement exceeds the proximity event threshold amount.

As mentioned above, any of these apparatuses and methods, setting the ambient temperature value during the device initialization stage may further include setting a wear-status state for the dental appliance to ‘not worn’ if either all of the one or more initial temperature readings are outside of the body temperature range, or if all of the one or more initial temperature readings are within the body temperature range and neither the change in the additional temperature readings has exceeded the temperature event threshold amount nor the change in the capacitance measurement has exceeded the capacitance event threshold amount, otherwise setting the wear-status state to ‘worn’.

The apparatuses or methods described herein may include setting a wear-status state for the dental appliance to a value of ‘not worn’ if both the ambient temperature value and the new temperature reading are outside of the body temperature range, and switching the value of the wear-status state if the ambient temperature value is within the body temperature range and the new temperature reading changes more than the temperature event threshold amount from one or more temperature readings taken immediately prior to the new temperature reading, or if the new temperature reading does not change more than the temperature event threshold amount but the change in the capacitance measurement from the capacitance sensor on the dental appliance exceeds the capacitance event threshold amount, otherwise leaving the wear-status state value the same if the ambient temperature value is within the body temp range, the new temperature reading does not change more than the temperature event threshold amount, and the change in the capacitance measurement does not exceed the capacitance event threshold amount.

These apparatuses may include any appropriate temperature sensor(s). For example, the temperature sensor may be one or more of: a thermistor, a resistance temperature detector (RTD), a thermocouples, and a semiconductor-based integrated sensors. In any of these apparatuses and methods the temperature (and/or capacitance or other corroborating sensor) readings may be accumulated over a window of readings (e.g., two or more readings, three or more readings, etc.) taken at a frequency (e.g., 10 Hz, 5 Hz, 1 Hz, 0.5 Hz, 0.1 Hz, etc.). The accumulated data may be averaged. For example, setting the ambient temperature value based on the one or more initial temperature readings may include setting the ambient temperature value based on an average of two or more initial temperature readings from the temperature sensor.

Also described herein are methods. In general, these methods may be performed by any of the apparatuses described herein and may be automated. For example, the methods described herein may include methods of determining ambient temperature and/or methods of determining one or more wear-status states (e.g., wear-status state). These method for determining wear-status state may also be referred to as methods of determining compliance.

For example a method of determining ambient temperature may include: setting an ambient temperature value from a temperature sensor on a dental appliance during a device initialization stage; updating, on an ongoing basis, the ambient temperature value using the temperature sensor on the dental appliance by comparing one or more new temperature readings from the temperature sensor to a body temperature range and to a temperature event threshold amount, and by comparing a capacitance measurement from a capacitance sensor on the dental appliance to a capacitance event threshold amount when the temperature reading is within the body temperature range and the temperature reading changes more than the temperature event threshold amount; and outputting the ambient temperature value.

Any of these methods may include updating the ambient temperature value by taking new temperature readings and comparing the new temperature readings and the ambient temperature value (e.g., the current, not-yet-updated ambient temperature value) to a body temperature range, and/or by comparing the new temperature reading to a temperature event threshold amount, and/or by comparing a corroboration data (e.g., capacitance) reading to a corroboration data event threshold amount (e.g., a capacitance event threshold amount). For example, updating the ambient temperature value may include setting the ambient temperature value to the new temperature reading if both the ambient temperature value and the new temperature reading are outside of the body temperature range, or setting the ambient temperature value to the new temperature reading if the ambient temperature value is within the body temperature range and the new temperature reading changes more than a temperature event threshold amount from one or more immediately prior temperature readings or if the new temperature reading does not change more than the temperature event threshold amount but a change in the capacitance measurement exceeds the capacitance event threshold amount, otherwise leaving the ambient temperature value the same.

In any of these examples a method may include setting an ambient temperature value during a device initialization stage based on one or more initial temperature readings from a temperature sensor of a dental appliance; updating the ambient temperature value by taking a new temperature reading from the temperature sensor and setting the ambient temperature value to the new temperature reading if both the ambient temperature value and the new temperature reading are outside of a body temperature range, or setting the ambient temperature value to the new temperature reading if the ambient temperature value is within the body temperature range and the new temperature reading changes more than a temperature event threshold amount from one or more immediately prior temperature readings or if the new temperature reading does not change more than the temperature event threshold amount but a change in a capacitance measurement from a capacitance sensor on the dental appliance exceeds a capacitance event threshold amount, otherwise leaving the ambient temperature value the same if the ambient temperature value is within the body temperature range, the new temperature does not change more than the temperature event threshold amount, and the change in the capacitance measurement does not exceed the capacitance event threshold amount; and outputting the ambient temperature value.

Any of the methods described herein may be methods of determining compliance, as mentioned above. Thus, any of these methods may track the wear-status state (“status state”) as either ‘worn’ or ‘not worn’. For example, a method of determining compliance may include: setting, during a device initialization stage, an ambient temperature value from the temperature sensor and setting a wear-status state for the dental appliance, wherein the wear-status state is either ‘worn’ or ‘not worn’; updating, on an ongoing basis, the ambient temperature value and the wear-status state using the temperature sensor by comparing one or more new temperature readings from the temperature sensor to a body temperature range and to a temperature event threshold amount, and by comparing a capacitance measurement from the capacitance sensor to a capacitance event threshold amount when the temperature reading is within the body temperature range and the temperature reading changes more than the temperature event threshold amount; and outputting the ambient temperature value.

In some examples the method may include: setting an ambient temperature value during a device initialization stage based on one or more initial temperature readings from a temperature sensor of a dental appliance and setting a wear-status state for the dental appliance, wherein the wear-status state is either ‘worn’ or ‘not worn’; updating the ambient temperature value by taking a new temperature reading from the temperature sensor and setting the ambient temperature value to the new temperature reading if both the ambient temperature value and the new temperature reading are outside of a body temperature range and setting the wear-status state to ‘not worn’, or setting the ambient temperature value to the new temperature reading and switching the state of the wear-status state, if the ambient temperature value is within the body temperature range and the new temperature reading changes more than a temperature event threshold amount from one or more immediately prior temperature readings or if the new temperature reading does not change more than the temperature event threshold amount but a change in a capacitance measurement from a capacitance sensor on the dental appliance exceeds a capacitance event threshold amount, otherwise leaving the ambient temperature value and the state of the wear-status state the same if the ambient temperature value is within the body temperature range, the new temperature does not change more than the temperature event threshold amount, and the change in the capacitance measurement does not exceed the capacitance event threshold amount; and outputting the ambient temperature value.

As mentioned, updating the ambient temperature value and the wear-status state may comprise setting the ambient temperature value to a temperature based on the new temperature readings if both the ambient temperature value and the new temperature reading are outside of a body temperature range and setting the wear-status state to ‘not worn’, or setting the ambient temperature value to a temperature based on the new temperature reading and switching the state of the wear-status state if the ambient temperature value is within the body temperature range and the new temperature reading changes more than a temperature event threshold amount from one or more immediately prior temperature readings or if the new temperature reading does not change more than the temperature event threshold amount but a change in a capacitance measurement from a capacitance sensor on the dental appliance exceeds a capacitance event threshold amount, otherwise leaving the ambient temperature value and the state of the wear- status state the same if the ambient temperature value is within the body temperature range, the new temperature does not change more than the temperature event threshold amount, and the change in the capacitance measurement does not exceed the capacitance event threshold amount.

The body temperature range may be, e.g., between about 32 and about 41 degrees Celsius (e.g., between about 30 degrees C. and about 42 degrees C., between about 31 degrees C. and about 41 degrees C., between about 32 degrees C. and about 40 degrees C., between about 33 degrees C. and about 39 degrees C.; between about 34 degrees C. and about 38 degrees C., between about 35 degrees C. and about 38 degrees C., between about 36 degrees C. and about 38 degrees C. etc.). The body temperature range may include the range of typically oral body temperature ranges (including high/fever body temperature ranges).

As mentioned above, any of these methods and apparatuses may include device initialization to set the initial values of the ambient temperature value, the state status, the body temperature range, and/or the event threshold amounts (e.g., temperature event threshold amount, capacitance event threshold amount, etc.). For example, setting the ambient temperature value during the device initialization step may include setting the ambient temperature value based on all or some of the one or more initial temperature readings when at least some of the one or more initial temperature readings are outside of the body temperature range. In some examples setting the ambient temperature value during the device initialization step comprises setting the ambient temperature value based on all of the one or more initial temperature readings when all of the one or more initial temperature readings are outside of the body temperature range. Setting the ambient temperature value during the device initialization step may comprise setting the ambient temperature value based on the one or more initial temperature readings that are outside of the body temperature range when at least one of the initial temperature readings are within the body temperature range. Setting the ambient temperature value during the device initialization step may include taking additional temperature readings from the temperature sensor, and setting the ambient temperature value based on the one or more initial temperature readings when all of the one or more initial temperature readings are within the body temperature range and either a change in the additional temperature readings exceeds the temperature event threshold amount or a change in the capacitance measurement from the capacitance sensor on the dental appliance exceeds the capacitance event threshold amount, alternatively setting the ambient temperature value based on the one or more initial temperature readings and the additional temperature readings after a predetermined device initialization time, when all of the one or more initial temperature readings are within the body temperature range and when neither the change in the additional temperature readings has exceeded the temperature event threshold amount nor the change in the capacitance measurement has exceeded the capacitance event threshold amount.

As mentioned above, updating the ambient temperature value comprises updating for a predetermined window of time. Any of these methods may include tracking the updates to the ambient temperature value over the same (or a different, e.g., shorter) predetermined window of time.

In general, outputting may comprise outputting the ambient temperature value as a stream of time-stamped data.

Any of these methods may include setting a wear-status state for the dental appliance to a value of ‘not worn’ if both the ambient temperature value and the new temperature reading are outside of the body temperature range, and switching the value of the wear-status state if the ambient temperature value is within the body temperature range and the new temperature reading changes more than the temperature event threshold amount from one or more temperature readings taken immediately prior to the new temperature reading, or if the new temperature reading does not change more than the temperature event threshold amount but the change in the capacitance measurement from the capacitance sensor on the dental appliance exceeds the capacitance event threshold amount, otherwise leaving the wear-status state value the same if the ambient temperature value is within the body temp range, the new temperature reading does not change more than the temperature event threshold amount, and the change in the capacitance measurement does not exceed the capacitance event threshold amount.

Setting the ambient temperature value based on the one or more initial temperature readings may include setting the ambient temperature value based on an average of two or more initial temperature readings from the temperature sensor. Any of these methods may include turning on a processor of the dental appliance to perform the device initialization step.

Also described herein are apparatuses comprising a dental appliance that includes: a dental appliance body forming one or more tooth-receiving cavities configured to be worn on a subject's teeth; and a sensing module coupled to the dental appliance body, the sensing module comprising: a temperature sensor; a corroborating sensor; one or more processors; and a memory coupled to the one or more processors, the memory storing instructions, that, when executed by the one or more processors, perform a method comprising: setting, during a device initialization stage, a wear-status state for the dental appliance, wherein the wear-status state is either ‘worn’ or ‘not worn’; switching the wear-status state when either or both of a change in a temperature reading from the temperature sensor exceed a temperature event threshold amount and/or a change in a corroboration sensor reading exceeds a corroboration event threshold amount; and outputting the wear-status.

Any of these apparatuses may be configured so that the method of the instructions further includes: setting, during the device initialization stage, an ambient temperature value and updating the ambient temperature value when the wear-status state switched from worn to not worn. Switching the wear-status state may include switching when the temperature reading from the temperature sensor is outside of a body-temperature range and when either or both of a change in a temperature reading from the temperature sensor exceed a temperature event threshold amount and/or a change in a corroboration sensor reading exceeds a corroboration event threshold amount.

The corroborating sensor may be an impedance sensor. In some examples, the corroborating sensor comprises one or more of: an impedance sensor, an optical sensor, a piezoelectric sensor, a force sensor, a voltage sensor, or a pressure sensor.

Setting during the device initialization step may include setting an ambient temperature value, further wherein switching the wear-status state comprises updating the ambient temperature value by recording a new temperature reading from the temperature sensor and setting the ambient temperature value to the new temperature reading if both the ambient temperature value and the new temperature reading are outside of a body temperature range and setting the status state to ‘not worn’, or setting the ambient temperature value to the new temperature reading and switching the state of the status state if the ambient temperature value is within the body temperature range and the new temperature reading changes more than a temperature event threshold amount from one or more immediately prior temperature readings or if the new temperature reading does not change more than the temperature event threshold amount but a change in a corroborating sensor measurement from the corroborating sensor exceeds a threshold amount, otherwise leaving the ambient temperature value and the state of the status state the same if the ambient temperature value is within the body temperature range, the new temperature does not change more than the temperature event threshold amount, and the change in the capacitance measurement does not exceed the capacitance event threshold amount.

In any of these apparatuses and methods, the corroborating sensor may be as a capacitance sensor. For example, the memory may be further configured to determine compliance with a treatment plan. In some cases the apparatus may be configured to determine compliance on a continuous or semi-continuous manner.

The sensing module may be configured to be removably coupled to the dental appliance body. In some cases the sensing module may comprises a base having a flange that is configured to engage with an opening through the dental appliance body.

Any of these sensing module may further comprise a force sensor and/or a pressure sensor. For example, the sensing module may comprise a force sensor configured to measure an amount of force exerted on a region of the dental appliance. Any of these apparatuses may include a pressure sensor configured to measure pressure changes within the intraoral cavity. In general, these apparatuses and methods may be configured to determine compliance based on a patterns in either (or both) force and pressure changes from biting, respiration, etc. Alternatively or additionally, these apparatuses may use a force sensor and/or pressure sensor to determine teeth clenching, bruxism, etc. For example, a pressure sensor may be used to determine clenching, bruxism, and sleep and/or respiratory disorders like sleep apnea. The methods and apparatuses described herein may include and/or may modify the sensors described in U.S. patent application Ser. No. 18/624,107, herein incorporated by reference in its entirety.

In any of these methods and apparatuses, outputting may comprise outputting the ambient temperature value as a stream of time-stamped data.

Also described herein are methods of determining ambient temperature from a dental appliance that include: determining if the dental appliance is worn or not worn in a mouth based on a temperature measured from a temperature sensor on the dental appliance, a change in the temperature measured temperature from the temperature sensor over time; setting an ambient temperature value to the temperature measured from the temperature sensor on dental appliance if the dental appliance is determined to be not worn; and outputting the ambient temperature value.

Determining if the dental appliance is worn or not worn in a mouth may be based on the temperature measured from a temperature sensor on the dental appliance, the change in the temperature measured temperature from the temperature sensor over time and a signal from a proximity sensor over time. For example, determining if the dental appliance is worn or not worn in a mouth may comprise comparing a temperature measured from a temperature sensor to a body temperature range, comparing the change in the temperature measured from the temperature sensor over time to a temperature activity threshold amount, and by comparing a change in the signal from the proximity sensor over time to a proximity activity threshold amount when the temperature measured from a temperature sensor is within the body temperature range and the change in the temperature measured from a temperature sensor changes more than the temperature activity threshold amount.

The proximity sensor may include a capacitance sensor. In some embodiments, the proximity sensor may include any suitable sensor that is used to measuring a parameter that can be used to infer physical proximity between the dental appliance and the subject's teeth or mouth, and may include one or more of a capacitance sensor, a pressure sensor, a force sensor, a voltage sensor, an optical sensor, etc.

Also described herein are apparatuses including a dental appliance that comprise: a dental appliance body forming one or more tooth-receiving cavities configured to be worn on a subject's teeth; and a sensing module coupled to the dental appliance body, the sensing module comprising: a temperature sensor; a corroborating sensor; one or more processors; and a memory coupled to the one or more processors, the memory storing instructions, that, when executed by the one or more processors, perform a method comprising: determining if the dental appliance is worn or not worn in a mouth based on a temperature measured from a temperature sensor on the dental appliance, a change in the temperature measured temperature from the temperature sensor over time; setting an ambient temperature value to the temperature measured from the temperature sensor on dental appliance if the dental appliance is determined to be not worn; and outputting the ambient temperature value.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A illustrates an example of a dental appliance including a sensing module as described herein.

FIG. 1B shows an example of a dental appliance including a dental appliance body and a sensing module.

FIG. 2 schematically illustrates an example of a sensing module as described herein.

FIG. 3A schematically illustrates one example of a method of tracking ambient temperature.

FIG. 3B schematically illustrates one example of a tracking use wear-status state (e.g., use in mouth) as described herein.

FIG. 4 is a flow diagram schematically illustrating one example of a device initialization portion of a method for tracking ambient temperature outside of the patient's body and tracking wear-status state.

FIG. 5 is a flow diagram schematically illustrating one example of ongoing monitoring using a method for tracking ambient temperature outside of the patient's body and tracking wear-status state.

FIGS. 6A-6B shows one example of testing results when ambient temperature values are below human body temperature and stable. FIG. 6B shows a magnified view of the sub-region B of FIG. 6A.

FIGS. 7A-7B shows one example of testing results tracking ambient temperature values when the ambient temperature is below human body temperature and cyclic. FIG. 7B shows an enlarged portion of FIG. 7A.

FIG. 8 shows an example of testing results when ambient temperature is within and over human body temperature.

FIGS. 9A-9B illustrate examples of sensing modules (e.g., sensor units) that are configured to lock into an oral appliance (e.g., a removable oral appliance).

FIGS. 10A-10B illustrate one example of a sensing module that is configured to removably lock into an oral appliance. FIGS. 10A shows a system including the sensing module and the oral appliance configured to removably receive the sensing module in a self-locking configuration. FIG. 10B shows the system of FIG. 10A assembled.

FIGS. 11A-11B illustrate examples of oral appliances including one or more removable/replaceable self-locking sensing module. FIG. 11A show a system including a sensing module coupled to an orthodontic retainer. FIG. 11B shows a system including a sensing module coupled to a palatal expander apparatus.

FIGS. 12A-12B illustrate an example of a system including a removable and/or replaceable sensing module and a dental aligner. FIG. 12A shows the system with the sensing module separate from the dental aligner, and FIG. 12B shows the system with the sensing module coupled to the dental aligner.

### DETAILED DESCRIPTION

The methods and apparatuses described herein use one or more temperature sensors and one or more corroborating (e.g., capacitance) sensors on a dental appliance to derive user compliance and/or other information about the user and the dental appliance, including other behavioral states. Any of these methods and apparatuses may use temperature data and in some cases proximity data (e.g., based on corroborating sensor measurements such as capacitance, optical measurements, etc.) to derive ambient temperature values. Ambient temperature values may provide useful information on their own, and may also be used to derive or interpret information about the user and/or the use of the dental appliance. In general, the methods described herein may apply one or more sources of the data, such as temperature data and proximity data, under different conditions in order to model ambient temperature.

The methods and the apparatuses for performing these methods may include a device initialization stage, during which the ambient temperature estimation model is initialized. The model may track ambient temperature and a wear-status state, indicating when the device is being worn. For example, at the method may begin by setting initial (start) values for the ambient temperature value and for the wear-status state (e.g., compliance or wear-status state). Following the initial wear-status state, the ambient temperature value and the wear-status state may be updated in an ongoing manner. The ambient temperature value (and optionally the wear-status state) may be output as a data stream that is updated as additional data is received. The data stream may include temperature and/or proximity (e.g., capacitance, optical, etc.) data.

In general, the methods and apparatuses for determining ambient temperature values may be used to determine the compliance status. The use of the ambient temperature values may simplify the analysis of the otherwise complication and difficult to interpret temperature and corroboration/proximity data and may provide accurate compliance information. In some cases the methods and apparatuses described herein may simplify the analysis of the temperature data from the one or more temperature sensors and the corroboration data (e.g., from a capacitance sensor or other corroborating sensor) by focusing on changes in the data and applying thresholds based on the context; this context may be provided by the derived ambient temperature value. In particular, the methods and apparatuses for performing them described herein may use the ambient temperature estimation model to reliably determine wear-status state even in cases (“corner cases”) when the ambient temperature is very close to the human body temperature, which may otherwise make it difficult or impossible to determine if the dental appliance is being worn using temperature, even with the use of corroboration data (e.g., electrical) information to determine contact, which may otherwise be difficult to interpret.

In general, the apparatuses described herein may include a sensing module that may be coupled or couplable to the body of a dental appliance, and may include a temperature sensor, an corroborating sensor (such as a capacitance sensor, a resistance sensor, an impedance sensor, etc.), a power supply, a communications module, and control circuitry including one or more processors and a memory. The memory may be coupled to the one or more processors and may include software, hardware and/or firmware that performs any of the methods for device initialization and ongoing or continuous tracking of ambient temperature (and optionally wear wear-status state). Optionally, the control circuity may be configured to perform post-processing including outputting the ambient temperature value (and in some examples wear wear-status state, or compliance).

The sensing modules described herein can be used in combination with various types of dental appliances to be worn in a user's mouth (intraoral appliances). The dental appliance may be an orthodontic appliance, such as an aligner or wire-and-bracket appliance, used to reposition one or more of the user's teeth to a desired arrangement, e.g., to correct a malocclusion. Alternatively or additionally, the intraoral appliance may be used to maintain one or more of the user's teeth in a current arrangement, such as a retainer. Other examples of intraoral appliances suitable for use in conjunction with the embodiments herein include sleep apnea treatment devices (e.g., mandibular advancement devices or splints), night guards (e.g., for treating bruxism), mouth guards, retainers, and palatal expanders.

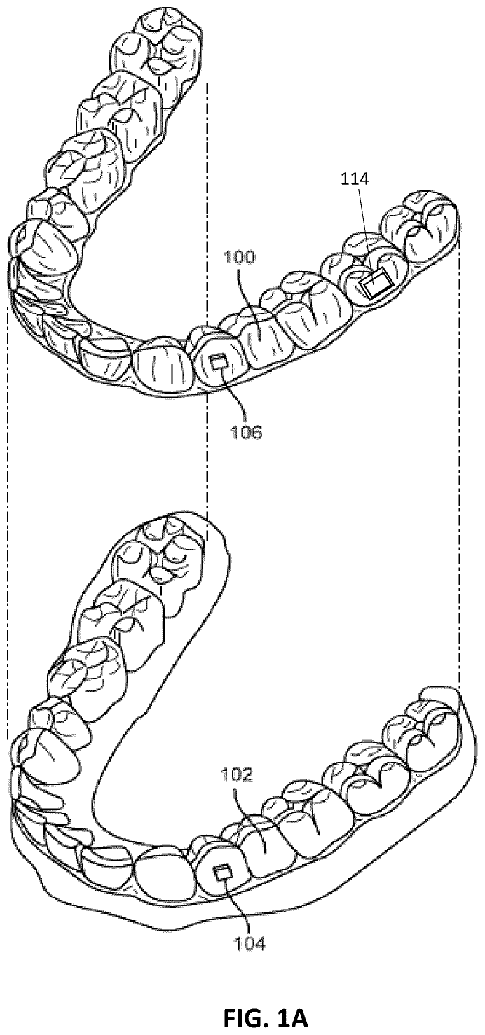

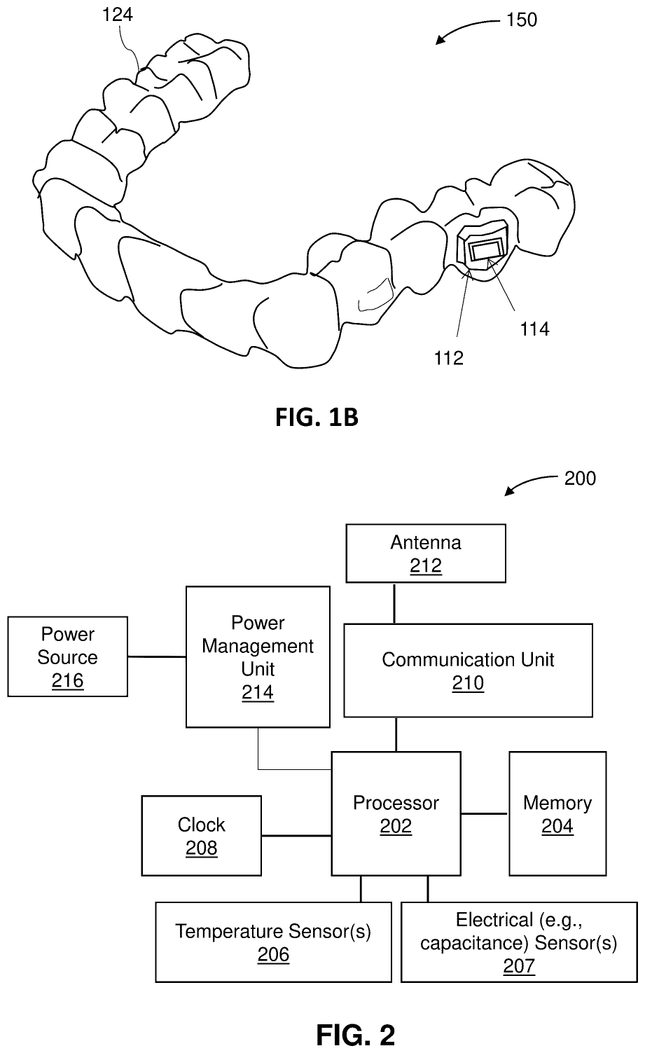

Appliances having teeth receiving cavities that receive and reposition teeth, e.g., via application of force due to appliance resiliency, are generally illustrated with regard to FIG. 1A. FIG. 1A illustrates an exemplary dental appliance, configured as a tooth repositioning appliance or aligner 100, that can be worn by a user in order to achieve an incremental repositioning of individual teeth 102 in the jaw. The appliance can include a shell having teeth-receiving cavities that receive and resiliently reposition the teeth, and a sensor module 114 attached thereto. An appliance or portion(s) thereof may be indirectly fabricated using a physical model of teeth. For example, an appliance (e.g., polymeric appliance) can be formed using a physical model of teeth and a sheet of suitable layers of polymeric material. In some embodiments, a physical appliance is directly fabricated, e.g., using rapid prototyping fabrication techniques, from a digital model of an appliance. Although the disclosure focuses on aligners, the disclosure contemplates the use of any suitable dental appliance (e.g., an aligner, a retainer, a mouth guard, a palatal expander) in the systems and methods described herein.

Although reference is made to an appliance comprising a polymeric shell appliance, the embodiments disclosed herein are well suited for use with many appliances that receive teeth, for example appliances without one or more of polymers or shells. The appliance can be fabricated with one or more of many materials such as metal, glass, reinforced fibers, carbon fiber, composites, reinforced composites, aluminum, biological materials, and combinations thereof for example. The appliance can be shaped in many ways, such as with thermoforming or direct fabrication (e.g., 3D printing, additive manufacturing), for example. Alternatively or in combination, the appliance can be fabricated with machining such as an appliance fabricated from a block of material with computer numeric control machining.

An appliance can fit over all teeth present in an upper or lower jaw, or less than all of the teeth. The appliance can be designed specifically to accommodate the teeth of the user (e.g., the topography of the tooth-receiving cavities matches the topography of the user's teeth), and may be fabricated based on positive or negative models of the user's teeth generated by impression, scanning, and the like. Alternatively, the appliance can be a generic appliance configured to receive the teeth, but not necessarily shaped to match the topography of the user's teeth. In some cases, only certain teeth received by an appliance will be repositioned by the appliance while other teeth can provide a base or anchor region for holding the appliance in place as it applies force against the tooth or teeth targeted for repositioning. In some embodiments, some, most, or even all of the teeth will be repositioned at some point during treatment. Teeth that are moved can also serve as a base or anchor for holding the appliance as it is worn by the user. Typically, no wires or other means will be provided for holding an appliance in place over the teeth. In some cases, however, it may be desirable or necessary to provide individual attachments or other anchoring elements 104 on teeth 102 with corresponding receptacles or apertures 106 in the appliance 100 so that the appliance can apply a selected force on the tooth. Exemplary appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the URL “invisalign.com”). Examples of tooth-mounted attachments suitable for use with orthodontic appliances are also described in patents and patent applications assigned to Align Technology, Inc., including, for example, U.S. Pat. Nos. 6,309,215 and 6,830,450, which are incorporated by reference herein in their entirety.

In some embodiments the sensing module may be integrated with a dental appliance. FIG. 2 schematically illustrates one example of a sensing module 200. Any of the components of the sensing module can be located on an appliance for the upper jaw, an appliance for the lower jaw, or a combination thereof. In some embodiments, the components of the sensing module may be distributed across multiple appliances in order to accommodate space limitations, accommodate power limitations, and/or improve sensing, for example. Additionally, some of the components of a sensing module can serve as a substrate for other components. FIG. 1B illustrates a sensing module 114 coupled to a first appliance 150 having a dental appliance body 124, to which the sensing module is attached 112. The first appliance 150 can be shaped to receive teeth of a user's upper arch or a user's lower arch. The system can include a sensing module 114 physically integrated with the first appliance 150. Alternatively, in some embodiments, the sensing module 114 may be releasably coupled to the first appliance, such that a user may attach or detach it easily. For example, a patient undergoing an orthodontic treatment with aligners (where a number of aligners are worn in sequence as the treatment progresses from stage to stage) may transfer the sensing module 114 to a next aligner in the sequence as the patient switches to the next stage in treatment.

Returning now to FIG. 2, the sensing module 200 includes a power source (e.g., battery) 216, and a power management subunit 214. The sensing module also includes a processor (e.g., CPU 202), one or more temperature sensors 206, one or more corroborating sensors (e.g., electrical sensor, such as a capacitance sensor 207) a memory (e.g., RAM 204 such as SRAM or DRAM; ROM such as EPROM, PROM, or MROM; or hybrid memory such as EEPROM, flash, or NVRAM), a communication unit 210 and antenna 212 and a clock 208.

The configuration of FIG. 2 can be varied. The sensing module 200 shown in FIG. 2 may be configured as an ECI. The sensing module can be implemented as an application-specific integrated circuit (ASIC) including one or more of the following components: a processor 202, a memory 204, one or more sensors (e.g., the temperature sensor(s) 206 and the electrical (e.g., capacitance) sensor(s) 207), a clock 208, a communication unit 210, an antenna 212, a power management unit 214, and/or a power source 216. The processor 202 (e.g., a central processing unit (CPU), microprocessor, field programmable gate array (FPGA), logic or state machine circuit, etc.), also referred to herein as a controller, can be configured to perform the various methods described herein. The memory 204 can be used to store instructions executable by the processor 202 to perform the methods provided herein. Additionally, the memory can be used to store sensor data obtained by the sensor(s) 206, 207 as discussed in greater detail below.

The sensing module 200 can include any number of sensors 206, 207 such as one, two, three, four, five, or more sensors. In some embodiments, the use of multiple sensors provides redundancy to increase the accuracy and reliability of the resultant data, since multiple sensors can be used to corroborate the data. Some or all of the sensors 206, 207 can be of the same type. Some or all of the sensors 206 can be of different types. Although the disclosure focuses on the use of a temperature sensor in conjunction with an electrical sensor, particularly a capacitance sensor, the disclosure contemplates the use of a temperature sensor in conjunction with any other corroborating sensor that measures a parameter correlated to proximity with a body tissue (e.g., tissues in the intraoral cavity). For example, the corroborating sensor may include one or more other sensor types such as: proximity sensors, audio sensors (e.g., microelectromechanical system (MEMS) microphones), color sensors (e.g., RGB color sensors), electromagnetic sensors (e.g., magnetic reed sensors, magnetometer), optical sensors, force sensors (e.g., force-dependent resistive materials), pressure sensors, motion sensors (e.g., accelerometers, gyroscopes), vibration sensors, piezoelectric sensors, strain gauges, pH sensors, conductivity sensors, gas flow sensors, gas detection sensors, humidity or moisture sensors, physiological sensors (e.g., electrocardiogramsors, bio-impedance sensors, photoplethysmography sensors, galvanic skin response sensors), or combinations thereof. In some embodiments, the proximity sensor may include any other suitable sensor that is used to measuring a parameter that can be used to infer physical proximity between the dental appliance and the subject's teeth or mouth, and may include one or more of a capacitance sensor, a pressure sensor, a force sensor, a voltage sensor, an optical sensor, etc. In some embodiments, the sensors herein can be configured as a switch that is activated and/or deactivated in response to a particular type of signal (e.g., optical, electrical, magnetic, mechanical, etc.). In some embodiments, the corroborating sensor may be a mechanical switch that is activated (e.g., mechanically pushed) when the dental appliance is worn by the patient. For example, the mechanical switch may be disposed on an interior surface of the dental appliance (i.e., facing a tooth surface) and may become activated when it is pushed against the tooth surface when the dental appliance is worn. As another example, the mechanical switch may be disposed on an exterior surface of the dental appliance (e.g., on a buccal side) and may become activated when it contacts an interior check surface. As another example, the mechanical switch may be disposed on an interior surface of the dental appliance (i.e., facing a tooth surface) and may become activated when it engages with an attachment on the tooth.

The temperature sensor(s) 206 and/or electrical (e.g., capacitance) sensor(s) 207 of the sensing module can be located with the sensing module at any portion of an intraoral appliance, such as at or near a distal portion, a mesial portion, a buccal portion, a lingual portion, a gingival portion, an occlusal portion, or a combination thereof. In some cases it may be helpful to position the temperatures sensor 206 and/or electrical (e.g., capacitance) sensor 207 near a tissue when the appliance is worn in the user's mouth, such as near or adjacent the teeth, gingiva, palate, lips, tongue, checks, airway, or a combination thereof. For example, when the dental appliance is worn, the temperature sensor(s) 206 can cover a single tooth, or a portion of a single tooth. Alternatively, the temperature sensor(s) 206 can cover multiple teeth or portions thereof. In embodiments where multiple sensors 206, 207 are used, some or all of the sensing module can be located on a buccal side or a lingual side of the dental appliance.

In any of the sensing modules described herein an analog-to-digital converter (ADC) can be used to convert analog sensor data into digital format, if desired. The processor 202 can process the sensor data obtained by the sensor(s) 206, 207 in order to determine appliance usage and/or user compliance, as described herein. The sensor data and/or processing results can be stored in the memory 204. Optionally, the stored data can be associated with a timestamp generated by the clock 208 (e.g., a real-time clock or counter).

The sensing module 200 may include a communication unit 210 configured to transmit the data stored in the memory (e.g., sensor data and/or processing results) to a remote device. The communication unit 210 can utilize any suitable communication method, such as wired or wireless communication methods (e.g., RFID, near-field communication, Bluetooth, ZigBee, infrared, etc.). The communication unit 210 can include a transmitter for transmitting data to the remote device and an antenna 212. Optionally, the communication unit 210 includes a receiver for receiving data from the remote device. In some embodiments, the communication channel utilized by the communication unit 210 can also be used to power the sensing module 200, e.g., during data transfer or if the sensing module 200 is used passively.

The remote device can be any computing device or system, such as a mobile device (e.g., smartphone), personal computer, laptop, tablet, wearable device, etc. Optionally, the remote device can be a part of or connected to a cloud computing system (“in the cloud”). For example, the remote device may be a server. The remote device can be associated with the user, the treating practitioner, medical practitioners, researchers, etc. In some embodiments, the remote device is configured to process and analyze the data from the sensing module 200, e.g., in order to monitor user compliance and/or appliance usage, for clinical and/or research purposes, and the like.

The sensing module 200 can be powered by a power source 216, such as a battery. In some embodiments, the power source 216 is a printed and/or flexible battery, such as a zinc-carbon flexible battery, a zinc-manganese dioxide printed flexible battery, or a solid-state thin film lithium phosphorus oxynitride battery. The use of printed and/or flexible batteries can be advantageous for reducing the overall size of the sensing module and avoiding user discomfort. For example, printed batteries can be fabricated in a wide variety of shapes and can be stacked to make three-dimensional structures, e.g., to conform the appliance and/or teeth geometries. Likewise, flexible batteries can be shaped to lie flush with the surfaces of the appliance and/or teeth. Alternatively or in combination, other types of batteries can be used, such as supercapacitors. In some embodiments, the power source 216 can utilize lower power energy harvesting methods (e.g., thermodynamic, electrodynamic, piezoelectric) in order to generate power for the sensing module. Optionally, the power source 216 can be rechargeable, for example, using via inductive or wireless methods. In some embodiments, the user can recharge the power source 216 when the appliance is not in use. For example, the user can remove the intraoral appliance when brushing the teeth and place the appliance including the sensing module on an inductive power hub to recharge the power source 216.

Optionally, the sensing module can include a power management unit 214 connected to the power source 216. The power management unit 214 can be configured to control when the sensing module is active (e.g., using power from the power source 216) and when the sensing module is inactive (e.g., not using power from the power source 216). In some embodiments, the sensing module is only active during certain times so as to lower power consumption and reduce the size of the power source 216, thus allowing for a smaller sensing module.

In some examples, the sensing module includes an activation mechanism (not shown) for controlling when the sensing module is active (e.g., powered on, monitoring appliance usage) and when the sensing module is dormant (e.g., powered off, not monitoring appliance usage). The activation mechanism can be provided as a discrete component of the sensing module, or can be implemented by the processor 202, the power management unit 214, or a combination thereof. The activation mechanism can be used to reduce the amount of power used by the sensing module, e.g., by inactivating the sensing module when not in use, which can be beneficial for reducing the size of the power supply 216 and thus the overall device size.

In some embodiments, the sensing module is dormant before being delivered to the user (e.g., during storage, shipment, etc.) and is activated only when ready for use. This approach can be beneficial in conserving power expenditure. For example, the components of the sensing module can be electrically coupled to the power source 216 at assembly, but may be in a dormant state until activated, e.g., by an external device such as a mobile device, personal computer, laptop, tablet, wearable device, power hub etc. The external device can transmit a signal to the sensing module that causes the activation mechanism to activate the sensing module and enter the device initialization stage. As another example, the activation mechanism can include a switch (e.g., mechanical, electronic, optical, magnetic, etc.), such that the power source 216 is not electrically coupled to the other components of the sensing module until the switch is triggered. For example, in some embodiments, the switch is a reed switch or other magnetic sensor that is held open by a magnet. The magnet can be removably attached to the sensing module, or may be integrated into the packaging for the sensing module or appliance, for example. When the sensing module is separated from the magnet (e.g., by removing the magnet or removing the device and appliance from the packaging), the switch closes and connects the power source 216. As another example, the sensing module can include a mechanical switch such as a push button that is manually actuated in order to connect the power source 216. In some embodiments, the activation mechanism includes a latching function that locks the switch upon the first actuation to maintain connectivity with the power source so as to maintain activation of the sensing module.

As mentioned, the methods and apparatuses described herein may include a device initialization stage, an ongoing (e.g., continuous, discrete, etc.) monitoring stage, during which the ambient temperature (and optionally, the wear-status state) may be tracked, and a post-processing stage.

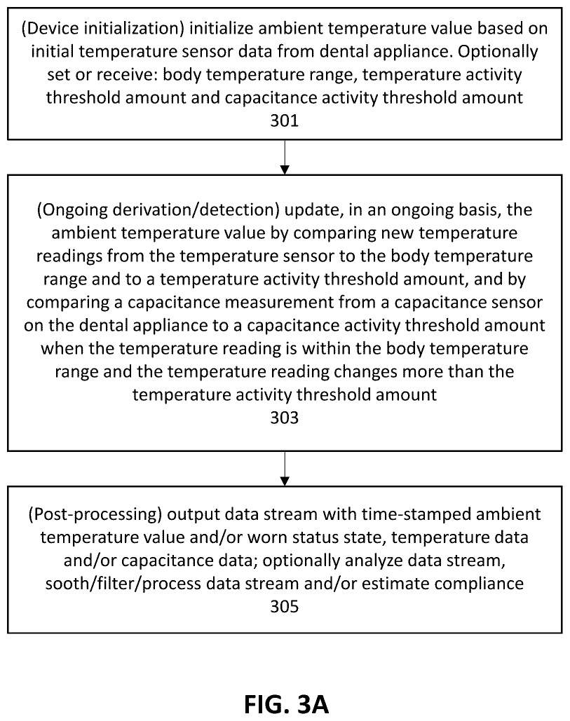

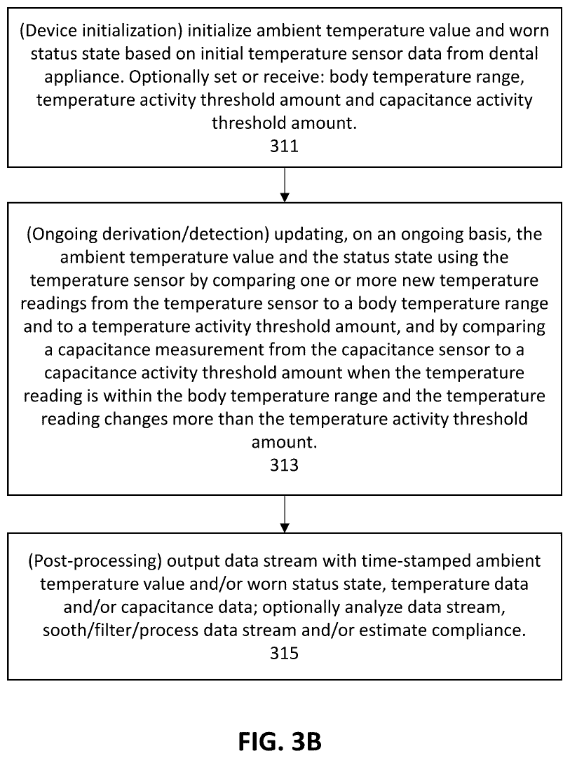

FIG. 3A illustrates one example of a method for determining ambient temperature. These apparatuses and methods may output ambient temperature and wear-state status, may output just ambient temperature, or may output just wear-state status. For example, in FIG. 3A, the schematically illustrated method includes a device initialization step of setting an ambient temperature value from a temperature sensor on a dental appliance during a device initialization stage at step 301. During step 301, the method may include initializing an ambient temperature value based on initial temperature sensor data from the dental appliance. Optionally, this step may set or receive a body temperature range, a temperature event threshold amount, and a capacitance event threshold amount. The method also illustrates the ongoing monitoring stage 303. In this example, the monitoring stage 303 may include updating, on an ongoing basis, the ambient temperature value using the temperature sensor on the dental appliance by comparing one or more new temperature readings from the temperature sensor to a body temperature range and to a temperature event threshold amount, and by comparing a capacitance measurement from a capacitance sensor on the dental appliance to a capacitance event threshold amount when the temperature reading is within the body temperature range and the temperature reading changes more than the temperature event threshold amount. The method illustrated in FIG. 3A may output (during the ongoing monitoring stage 303 and/or during the post-processing stage 305) a data stream including the ambient temperature value(s), capacitances, and/or directly measured temperatures. The data stream values may be time-stamped.

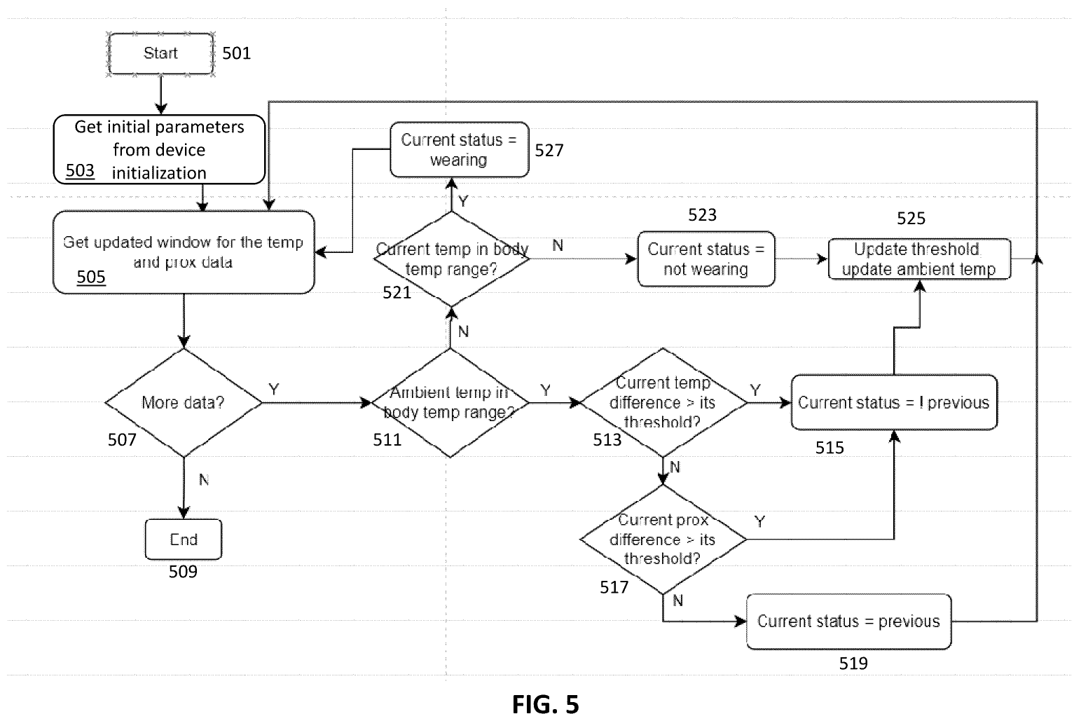

Any of these methods may include determining ambient temperature values and/or wear-status state, as illustrated in FIG. 3B. The method shown in FIG. 3B is similar to that of FIG. 3A, but outputs both ambient temperature values and wear-status state. In some examples however, the method of FIG. 3B may instead output just wear-status state. In general, multiple parameters may be established during a device initialization stage 311, such as the initial values of ambient temperature, the initial threshold/limit of the data difference. These initial values may act as a range regulator for the monitoring stage 313. In any of these methods the ongoing monitoring stage may involve continuous or semi-continuous monitoring, periodic monitoring, or variable monitoring. In some examples, variable monitoring may be performed at particular times or frequencies based on a predictive schedule. For example, the monitoring steps may be performed less frequently when the user is expected to be sleeping (e.g., between the hours of midnight and 6 am), during time periods where the wear-state status has historically remained unchanged, and/or other time periods determined based on analyzing historical data. The post-processing stage 315 includes outputting a data stream with the wear-status state data, which may also be time stamped. As will be described in the example shown in reference to FIG. 5, below, monitoring stage 313 may be configured to so that different decisions may be made under different circumstances, in comparison with the event threshold values and the body temperature range, and the event threshold values and/or body temperature range could also be updated dynamically under the determined conditions. The output of the post-processing stage 315 may provide the wear-status state (e.g., wearing/not wearing) for the corresponding time stamp. In some examples this post-processing stage 315 may be performed of the status buffer to smooth the results and get rid of some abnormality in some of the occurrence. Finally, the result output may contain a list or array of the conclusive data to indicate the overall compliance information for the given time period.

The output data stream may include directly measured temperatures, capacitances, and/or derived ambient temperatures over the course of the monitoring, including time-stamps. In some examples the post-processing output data stream may refine the directly measured data, for example, processing by smoothing and/or filtering. Processing may also include analyzing the data to identify potential environmental information from the data, such as identifying periods when the dental appliance is being worn in the oral cavity or is not being worn, periods where the person wearing the dental appliance is drinking, periods where the person wearing the dental appliance is sleeping, etc. In some examples output may be limited to the post-processing output to save memory and/or bandwidth. In some examples, both the post-processing data stream and ongoing monitoring data stream may be output.

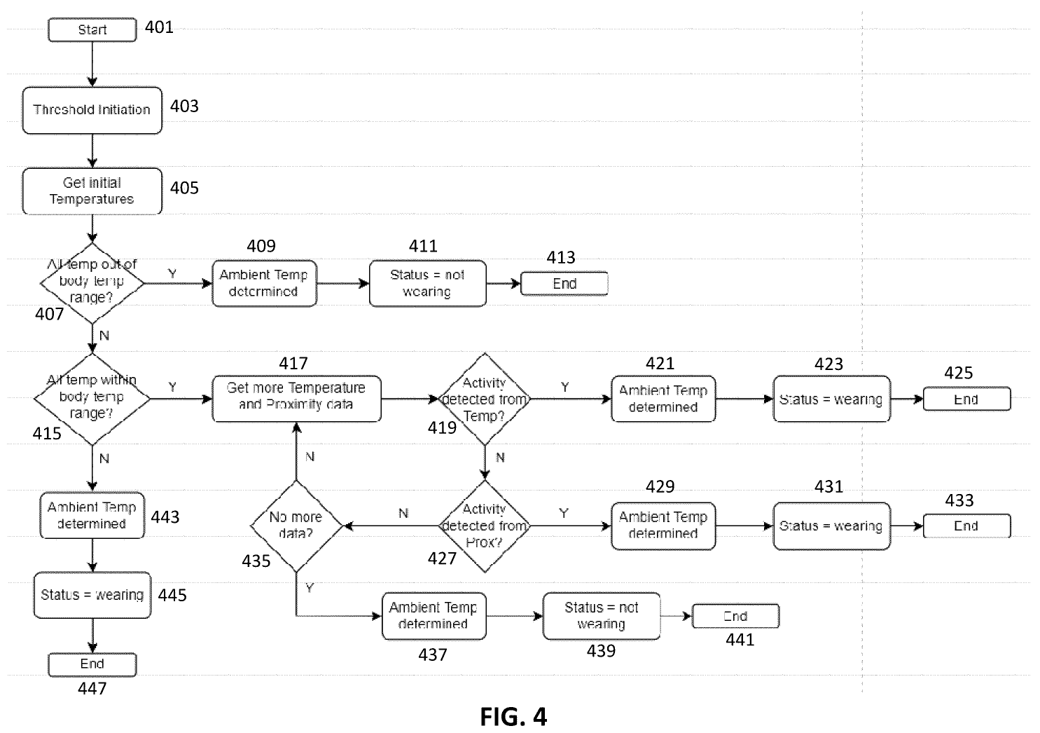

FIG. 4 illustrates one example of a method of calibration that may be performed by any of the apparatuses or as part of a method described herein. In FIG. 4 the calibration may start at step 401 when, e.g., the device is turned on and/or restarted. At initialization step 403, event thresholds (e.g., temperature event threshold amount, capacitance event threshold amount, etc.) may be initialized. The initialization step may also include initialization of the body temperature range. Body temperature range may be set, for example, based on population data (e.g., based on measured oral temperatures, e.g., between 35° C. and 38° C.). The buffers to store the current ambient temperatures values and/or wear-status state(s) may also be established. The initial ambient temperature value may be determined using the method illustrated in FIG. 4, based on initial temperatures acquired at step 405 (e.g., between 1-5 readings, e.g., 2 readings, 3 readings, 4 readings, 5 readings, etc.). The initial temperatures may be acquired, for example, by one or more temperature sensors on the device within a specific time window (e.g., a predetermined time window) after the device is turned on and/or restarted. Based on the initial temperature readings and the initialized parameters, the method may determine if the initial ambient temperature value should be adjusted and/or if the wear-status state should be switched, e.g., from wearing to not wearing.

In some examples, the initial ambient temperature reading may be deduced based on the initial temperature reading(s) and/or initial capacitance reading(s), which in turn may be used to deduce the likely wear-status state of the apparatus. For example, if the initial temperature readings are all outside of the body temperature range it is unlikely to be within an oral cavity and thus likely that the user is not wearing the dental appliance at the time of these temperature readings; in some cases the user may be switching from not wearing the dental appliance to wearing the dental appliance, or may be switching from wearing the dental appliance to not wearing the dental appliance. However, if the initial temperature readings are within the body temperature range, it is not immediately conclusive that the dental appliance is being worn. This is because temperature readings within the body temperature range may be due to the user wearing the dental appliance, or may be because the actual ambient temperature is within the body temperature range (e.g., temperatures on a hot day or temperatures in a hot car might reach, e.g., 97 degrees Fahrenheit/36.1 degrees Celsius). Thus, if the initial temperature readings are within the body temperature range, temperature alone is not sufficient to determine the actual ambient temperature (or to deduce if the dental appliance is being worn). Thus, in these cases the method or apparatus may use the event thresholds to determine ambient temperature and wear-status state. As shown in FIG. 4, the initial temperature readings may be compared to the body temperature range 407, 415. If all of the initial temperature readings are outside of the body temperature range 407, then the initial ambient temperature value may be set to one or more of the initial temperature readings 409. For example, the initial ambient temperature value may be set to an average of the initial temperature readings. Similarly, the wear-status state may be set to “not wearing” the dental appliance 411, and the device initialization stage may end 413. If only some of the initial temperature readings are outside of the body temperature range 415, then it may be determined that the user is inserting or withdrawing the dental appliance, and the initial ambient temperature value may be set to one or more of the initial temperature readings that are outside of the body temperature range 443; for example, the initial temperature readings may be set to the average of the two or more initial temperature values that are outside of the body temperature range. The wear-status state may be initially set to ‘wearing’ 445 and the device initialization stage may end 447.

However, if all of the initial temperature readings 405 are within the body temperature range, then one or more additional temperature readings may be taken 417, and the one or more additional temperature readings may be compared to the temperature event threshold amount 419 to determine if the temperature has changed or is in the process of changing, which may indicate that the dental appliance is being placed into or taken out of the mouth. When the comparison determines that the temperature is in the process of changing (e.g., the change in the temperature between sequential readings exceeds the temperature event threshold amount) then the initial ambient temperature value may be set to one or more (in some examples, the average) of the initial temperature readings 421, and the wear-status state may be set to indicate ‘wearing’ 423 and the device initialization step may end 425. For example, in some cases the temperature event threshold amount may be about 0.5 degrees C., about 1 degree C., about 1.5 degrees C., about 2 degrees C., about 2.5 degrees C., about 3 degrees C., about 3.5 degrees C., about 4 degrees C., about 4.5 degrees C., about 5 degrees C., etc.). The temperature event threshold may be preset (predetermined) or set by a user. In general, the event thresholds (including the temperature event threshold and/or the capacitance event threshold, etc.) may be adjusted to adjust the sensitivity of the apparatus or method.

If the change between sequential temperatures (from the additional, and optionally initial, temperatures) does not exceed the temperature event threshold amount, then the apparatus performing the method may use an indicator of proximity to the intraoral cavity or tissues of the intraoral cavity, such as capacitance (or another corroborating sensor), to determine if there has been a change indicating that the dental appliance is changing from being worn to not worn or from not being worn to being worn 427. For example, the method may include comparing difference in capacitance readings between the new capacitance reading(s) (which may be taken at or shortly after the new temperature reading(s)), or between the new capacitance reading(s) and the prior capacitance reading, which may have been taken at the same time as the initial temperature readings. If the change in capacitance reading activity (e.g., the change in capacitance) is greater than the capacitance event threshold amount, then the apparatus may set the ambient temperature value to one or more of the initial temperature readings (e.g., an average of the initial temperature readings) 429, and the wear-status state may be set to ‘wearing’ 431 and the device initialization step may end 433. If no proximity activity is detected, e.g., the change in capacitance between sequential capacitance measurements is less than the capacitance event threshold amount 435, then the apparatus performing the method may take one or more additional temperature readings (and capacitance readings) 417 and repeat the process of detecting activity from the temperature and capacitance outlined above. This process may be repeated a predetermined or adjustable number of times until a limit (e.g., twice, three times, four times, five times, six times, etc. reflecting a window size) is reached, after which the ambient temperature value may be set to one or more of the initial and/or additional temperature readings (e.g., in some cases an average of all or some of these) 437 and the wear-status state may be set to ‘not wearing’ 439 and the device initialization stage may end 441. Thus, during the ambient temperature initialization, the ambient temperature value and one or more wear-status states may be set to an initial status, and a wear-status is assigned to the device as either wearing, or not wearing. In some examples the apparatuses and methods described herein may use a capacitance sensor to determine if the dental appliance is in proximity to the oral tissue. A capacitance sensor may use capacitive coupling to detect a chance in the dielectric properties of the material in contact with the sensor. Thus the capacitive event threshold may be set to (preset) to reflect a change in capacitance from air and/or saliva to tissue (e.g., gingiva/teeth, etc.). Thus, as the capacitance event threshold may be set or selected to indicate a change from wearing and not wearing the dental appliance.

Although the disclosure focuses on using capacitance to measure proximity, the proximity sensor may measure other signals such as optical (e.g., infrared, visible wavelength LED) signals from a receiver-emitter pair (e.g., an infrared emitter on the dental appliance and a receiver on an attachment bonded to the patient's teeth), an electrode pair (e.g., a first electrode on the dental appliance and a second electrode on an attachment bonded to the patient's teeth), a voltage sensor). Thus, the proximity threshold amount may be an threshold specific to the proximity sensor type (e.g., optical signal, such as IR, visible light, etc.).

After the end of the device initialization stage the apparatus performing the method may perform ongoing monitoring, as illustrated schematically in FIG. 5. For example, following device initialization, the method may use the initial parameters and may run a continuous monitoring technique for a window of time, or an open-ended period of time. As described previously, the ongoing monitoring may involve continuous or semi-continuous monitoring, periodic monitoring, or variable monitoring. The values of these initial parameters, such as the ambient temperature value and wear-status state(s) may be updated as needed to increase the sensitivity for an adaptive real time monitoring. The changes may be recorded as a time-stamped data stream. In any of these apparatuses and methods, the monitoring for each cycle may include buffering a certain period of time (e.g., for two temperature and/or proximity readings, for three temperature and/or proximity readings, for four temperature and/or proximity readings, for five temperature and/or proximity readings, etc.). The most up-to-date ambient temperature value as well as the current temperature and proximity data may be used.

For example, FIG. 5 illustrates a process flow that may be used for continuous tracking of ambient temperature (and/or compliance). In FIG. 5, continuous tracking may begin 501 by accessing the initial parameters from the device initialization 503, including the initial ambient temperature value, initial wear-status state(s), thresholds, and ranges (e.g., body temperature range). One or more additional readings, e.g., temperature readings and/or corroboration data (e.g., compliance/proximity) readings may be taken 507. In some examples the process may include a step of determining how long to perform the continuous monitoring for, by setting a window size 505 or by indicating that it should be continued until stopped. Thus, the process may be continued until the window is completed 509 or until stopped, e.g., by a user.

During each cycle, one or more temperature readings (and in some cases proximity/capacitance readings) are taken and the ambient temperature value, e.g., the current ambient temperature value which may be set to the initial ambient temperature valve from the device initialization stage at the start of the continuous tracking, may be compared to the body temperature range 511. If the ambient temperature value (the “current” ambient temperature value) is outside of the body temperature range, the current one or more temperature reading(s) may be compared to the body temperature range 521, and if they are within the body temperature range, then the ambient temperature value is unchanged, and the wear-status state may be set to the ‘wearing’ 527 and the cycling is repeated 505.

If the current one or more temperature reading(s) are outside of the current body temperature range, then the wear-status state will be updated to set to ‘not wearing’ 523 and the ambient temperature value may be set to the current temperature reading 525.

If the current ambient temperature is within the body temperature range 511, the apparatus performing the method may examine a change in the temperature from the most recent temperature readings and/or a change in the proximity (e.g., based on an corroboration data reading, such as capacitance) to determine if the dental appliance is being worn or not. The apparatus may first compare the change in the temperature reading, relative to one or more prior temperature readings, to the temperature event threshold amount 513. If the change in temperature is greater than the temperature event threshold amount, the apparatus may deduce that the apparatus is being placed into or taken out of the user's mouth, and may set the value of the wear-status state by flipping the current value of the wear-status state 515. If the current value of the wear-status state is set to ‘worn’, the wear-status state may instead be set to a new value of ‘not worn.’ Similarly, if the current value of the wear-status state is set to ‘not worn’, the wear-status state may instead be set to a new value of ‘worn.’ If the change in the temperature reading does not exceed the temperature event threshold amount 513, then the apparatus performing the method may then compare a current indicator of proximity data, such as capacitance, e.g., by determining if a change in the capacitance reading exceeds the capacitance event threshold amount 517. The change in the capacitance reading may be determined as the difference between the current capacitance reading and one or more prior (e.g., immediately prior) capacitance readings. If the change in the capacitance reading exceeds the capacitance event threshold amount then the apparatus performing the method may deduce that the apparatus is being placed into or taken out of the user's mouth, and may flip the current value of the wear-status state 515. The ambient temperature value may then be updated, e.g., may be set to the current temperature value. In some examples the threshold (e.g., the temperature event threshold amount) may also be updated. For example, the temperature event threshold amount may be scaled based on the magnitude of the difference between the ambient temperature, e.g., the new ambient temperature value, and either the current temperature reading or a body temperature amount.

If the change in the temperature readings does not exceed the temperature event threshold amount 513 and the change in the capacitance readings does not exceed the capacitance event threshold 517, then the apparatus performing the method may conclude that neither the ambient temperature nor the wear-status state have changed and will leave them unchanged 519.

As discussed above, this method may be repeated continuously until either the window of time has been completed or the process is manually stopped. While the process is ongoing, the values of the ambient temperature value, the wear-status state, and optionally the temperature sensor readings (and/or corroborating sensor readings) may be recorded along with time information. This information may be stored as a data stream. Thus, any of these apparatuses and methods may perform post-processing, forming and/or modifying the data stream or other collected data. For example, after the continuous monitoring in the steps above, the output stream including the values of the wear-status states and/or ambient temperatures may be timestamped. The data may be filtered, compressed, and/or otherwise edited. For example, post processing may be used to identify and/or smooth erratic regions of wear-status state values such as isolated short-term wearing status, which may not accurately reflect a users' actual practice.

### EXAMPLES

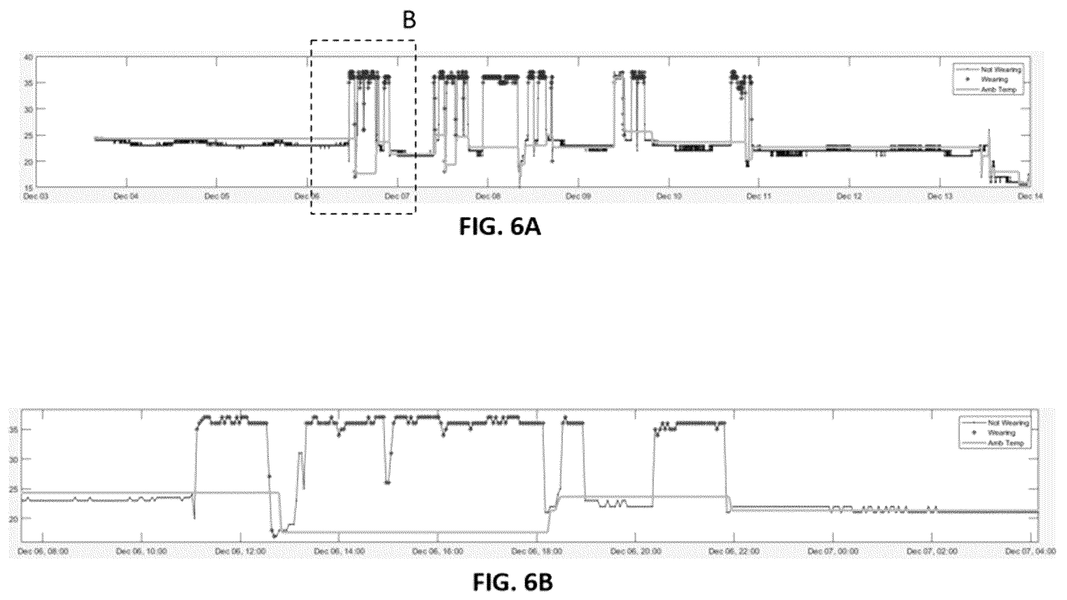

An example apparatus performing the method described above, similar to that shown schematically in FIGS. 1A-1B and 2, was used and tested. The test apparatus was configured as shown in FIGS. 4 and 5. FIGS. 6A and 6B illustrate one example of the data stream collected when the ambient temperature was lower than the body temperature range (set to a human body temperature range, such as between about 32 degrees C. and 38 degrees C.). The ambient temperature in this example is relatively stable, and this test lasted for more than 10 days. The periods when the dental appliance were being worn are indicated by asterisks. FIG. 6B shows a magnified view of sub-region of FIG. 6A (subregion “B” in FIG. 6A). FIG. 6A shows the overall 10 days curve, and FIG. 6B shows the data for a single day. In FIGS. 6A-6B, the y-axis shows temperature values in Celsius.

As shown in FIGS. 6A-6B, the apparatus was able to distinguish between periods of compliance (when the dental appliance was worn in the mouth) and periods during which the dental appliance was not being worn.

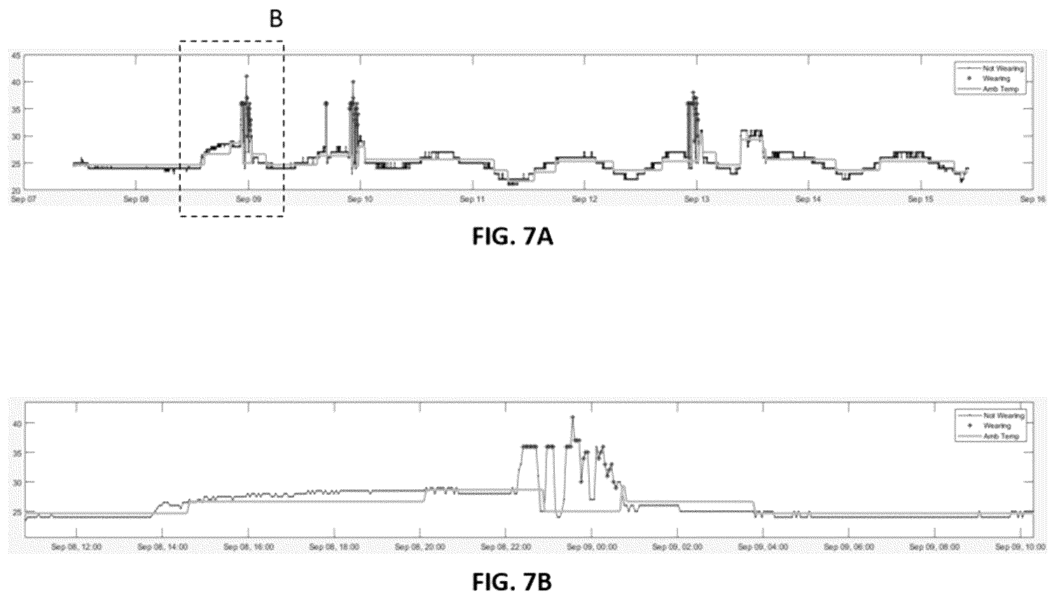

FIGS. 7A-7B illustrate another example of a data stream from the test apparatus when the ambient temperature is less than the body temperature range but has a periodical cycle during day and night. FIG. 7B shows a magnified view of the boxed region (“B”) of FIG. 7A. In this example the ambient temperature value was dynamically updated as part of the process. The cyclic patterns of the ambient temperatures were successfully detected, providing a reliable reference for the monitoring in real time. In addition, wear-status state monitoring correctly indicated when the apparatus was worn or not worn.

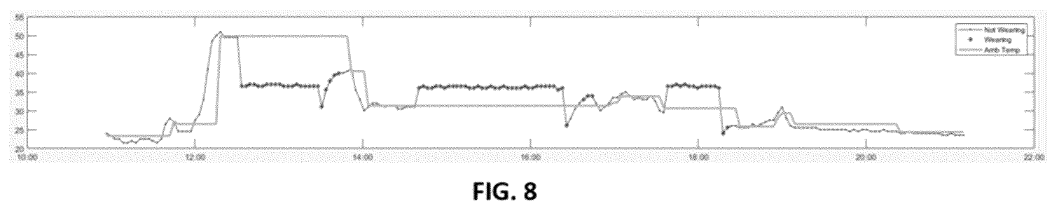

FIG. 8 shows another example, in which the ambient temperature was within the body temperature range over at least a portion of the testing period (e.g., between 14 and 18 hours), and was above the body temperature region during another portion (e.g., between 12 and 14 hours). In this example the test was conducted in a car, in which the cabin reached a relatively high temperature. The results show that the ambient temperature generated by this process was able to respond to the cabin temperature changes in a real-world scenario. The method also successfully deduced wearing/not wearing of the dental appliance in this scenario.

In addition to detecting wearing/not wearing of the dental appliance, these methods and apparatuses may also be used to detect and/or monitor other general patient health states, such as body temperature and respiration, when the user is drinking and/or eating, when the user is cleaning the dental appliance, and/or changes in body temperature.

For example, these apparatuses may be configured to interpret changes in the ambient temperature that reflect changes in the patient's body temperature and/or respiration. The method or apparatus may provide an indicator for general health (e.g., exercise, wellness, etc.) or for clinically relevant health status (e.g., detecting a disorder or disease such as a respiratory and/or cardiac disorder).

In any of these methods and apparatuses, the sensing module may be releasably or replaceably connected to an oral device. In addition, any of the oral sensing devices described herein may be configured as stand-alone devices that can be attached to and removed from a structure inside the mouth (tooth, implant, gum, palate, etc.). In some examples the sensing module is, or is part of, an oral sensing device that can be clipped onto or wrapped around a tooth. In particular, these apparatuses may be configured so that the sensing module and/or the oral appliance have one or more mating features so that the sensing module can be easily coupled with (e.g., snapped onto) the oral appliance for secure placement. The sensing modules described herein may configured as a sealed sensing module or and may include any of the features or techniques described in U.S. patent application Ser. No. 18/396,679, tiled “APPARATUS AND METHODS FOR EXTENDED INTRAORAL BODY TEMPERATURE MONITORING” and filed on Dec. 26, 2023, herein incorporated by reference in its entirety.

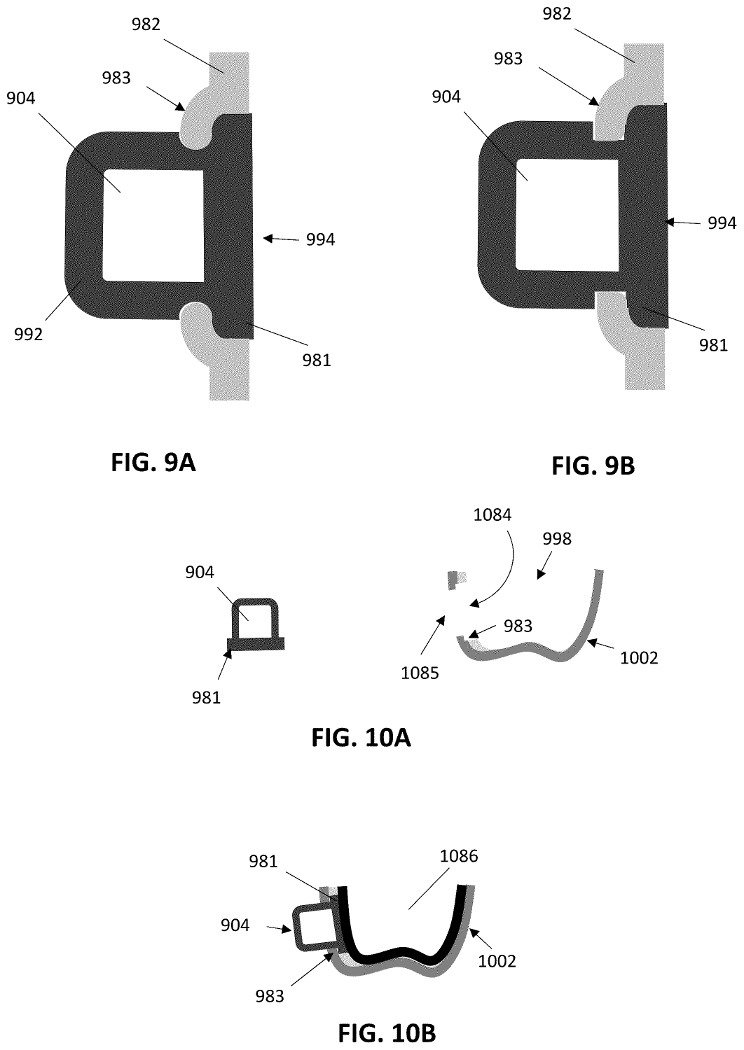

For example, the sensing module can be removably attached to an oral device or to a mount that is worn on the teeth (or a tooth) so that the sensing module can be replaced with another unit as needed or modified before recombining the sensing module with the device or mount. Removable/replaceable and self-locking sensing modules are described in greater detail below in reference to FIGS. 9A-9B and 10A-10B, but may generally include a sensing module that may be releasably connected to an oral device or to a mount (e.g., intraoral mount), an in particular may be configured to self-lock in position. An oral device (also referred to equivalently as an intraoral device) may include, but is not limited to, an aligner, retainer, palatal expander, or any other oral/intraoral device. This may allow the same sensing module to be used with different intraoral devices or intraoral mounts, and/or may allow multiple different sensing modules to be used with the same intraoral mount or device.

For example, the sensing module may be removably attached to an oral device. The oral device may be one (or a series) of dental appliance that are configured to be worn on the subject's teeth and may exert a force or forces to control the position and/or orientation of the teeth, or in some cases the palate (e.g., with a palatal expander) or a mandible. For example, FIGS. 9A-9B, 10A-10B, 11A-11B and 12A-18B show examples of a sensing module that may be releasably connected to an oral device. These examples may provide for the use of the same sensing module across multiple oral devices, such as a series of dental aligners used for orthodontic treatment. This may provide cost savings as the number of sensing modules needed may be reduced. Removable sensing modules may also be advantageous in cases where the associated appliance needs to be cleaned in a manner that would otherwise damage the sensing module. In some cases it may be particularly beneficial to use the same sensing module within the subject's oral cavity with different appliances, as this may avoid the need for calibration. In some cases multiple sensing modules may be swapped into the same oral device, or the same sensing module may be removed, modified, and replaced. This may make it easier to change the power supply (e.g., battery), sensors/sensor components, and/or repair the unit. In some cases one unit may be removed to recharge the power source (e.g. battery, capacitor) while another unit may be swapped into the oral device.

For example, FIGS. 9A-9B illustrate examples of a removable sensing module that may be coupled to an oral device. This configuration may be referred to as a self-locking design of the sensing module, as it is configured to couple with an opening in an oral device and be retained securely to the oral device until it is desired to separate the two. In FIG. 9A the sensing module 904 includes a housing 992 that may at least partially enclose one or more sensors (e.g., a thermal sensor, a pressure sensor, a capacitance sensor) and a processor that is configured to receive sensor data (e.g., temperature, pressure, capacitance) from the sensors, and to store, transmit, or store and transmit the sensor data as described herein. The housing includes a flange region at the base 994 of the sensing module. The flange region may be configured to mate with, and in FIGS. 9A and 9B, engage with (e.g., “snap into”) the oral device 982. In any of these examples the oral device may include an opening into which the region of the housing proud of the flange may extend through. This region may have an outer diameter that is approximately the same as or less than the inner diameter of the opening. The opening may include a receiving seat or lip region 983 around the opening that may engage with the housing of the sensing module. In FIG. 9A the opening on the oral device including a lip region 983 that snaps into a channel on an upper surface of the flange 981 to secure the sensing module to the oral device. In this case, the surfaces of these regions (e.g., the flange and/or channel on the outside of the sensing module and the lip region or receiving seat of the oral device) may be configured to seal against each other when the two are coupled together as shown.

Alternatively in FIG. 9B the flange 981 and/or channel on the outside of the sensing module and the lip region 983 or receiving seat of the oral device opening may mate together with a larger tolerance, as shown. In practice, in either FIGS. 9A-9B, mating the sensing module to the oral device may secure the two together so that they do not separate when inserting and removing the oral device to/from oral cavity and onto the teeth. Because the flange is located on the inside of the tooth-receiving region of the oral device, when the device is worn, the base 994 of the sensing module may prevent the sensing module from disengaging from the opening of the oral device by one or more teeth within the tooth-receiving cavity. Thus, the flange region may be oversized as compared with the opening (e.g., having a diameter that is greater than the diameter of the opening).

FIGS. 10A-10B illustrate one example of a sensing module 904 including a flange 981 on the base of the housing of the sensing module engaging with an opening 1085 of an oral device, configured as an aligner 1002 in this example. A tooth-receiving region 998 of the aligner is shown in FIG. 10A, and the opening 1085 through the lateral side (e.g., in this example, a buccal side) includes an inner lip region 983 that may engage with the upper surface of the flange 981 or a channel (not shown), as described in FIGS. 9A-9B. In use, the sensing module may be inserted (as illustrated by arrow 1084) from the tooth-receiving cavity 998 so that it extends out of the aligner 1002, as shown in FIG. 10B. In this example a tooth 1086 is shown within the tooth-receiving region of the aligner, and the self-locking sensing module is held in place and prevented from disengaging with the oral device by the tooth. As shown in this example, the base 994 of the sensing module is flush with (or approximately flush with, and in some cases may be slightly recessed relative to) the inner surface of the tooth-receiving region. This may prevent the sensing module from exerting unintended forces on the teeth.

Thus, the sensing module may be removed and replaced by removing the oral device from the mouth and applying force (e.g., pushing in towards the tooth-receiving region on the sensing module) to disengage the sensing module from the oral device. The oral device shown in FIGS. 10A-10B is an aligner; any appropriate oral device may be used, and may be formed from a polymeric material. In some cases the intraoral appliance is a thermoformed material; alternatively the intraoral appliance is formed (including the opening for coupling to the sensing module) by a direct fabrication technique (e.g., 3D printing).

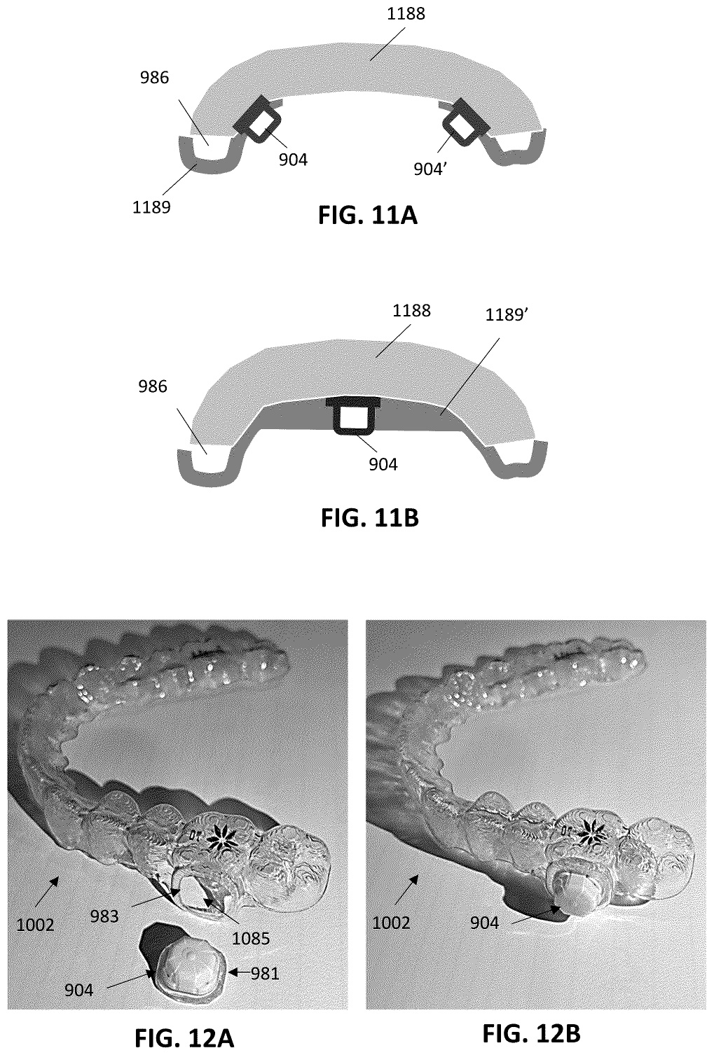

FIGS. 11A-11B show examples in which the oral device is a retainer 1189 and a palatal expander 1189′, respectively. These oral devices may otherwise be very similar to the aligner shown in FIGS. 10A-10B. In FIG. 11A the oral device (retainer 1189) includes a tooth-receiving region holding a tooth 986 and includes a region that extends from the lingual side towards the subject's palate 1188. In FIG. 11A the apparatus includes a pair of sensing modules 904, 904′ that are held within opening on the lingual side of the oral device. In FIG. 11B the oral device is a palatal expander 1189′ that also includes a tooth-receiving region on either side, shown engaged with teeth 986. In this example the sensing module 904 is engaged with and extend through the palatal region of the device. The opening through the oral device for holding the sensing module is located on the central region of the palatal region of the oral device. When the palatal expander is worn by the subject the base of the sensing module may be flush with the palatal region and the sensing module is prevented from separating form the oral device by the flange and the orientation of the sensing module relative to the palate (e.g., the sensing module flange is sandwiched between the palatal region of the oral device and the subject's palate). Although the sensing modules are depicted as being positioned in particular regions within the figures herein, the disclosure contemplates that the sensing modules may be positioned in any suitable location. For example, a sensing module of an aligner may be positioned on a lingual side (similar to the retainer in FIG. 11A). Similarly, a sensing module of a retainer may be positioned on a buccal side (similar to the aligner of FIGS. 10A-10B). Likewise, a sensing module of a palatal expander may be on a lingual or buccal side (alternatively or in addition to the palatal region).

FIGS. 12A-18B illustrate an example of an aligner 1002 shown before (FIGS. 12A) and after (FIG. 12B) insertion of the sensing module 904 including a housing and flange region 981 around the base (not visible) of the housing. The aligner 904 includes an opening 1085 having a lip or rim region 983 that is slightly recessed on the inside region of the opening (e.g., the tooth-receiving side of the aligner).

In addition to the self-locking removable sensing modules described above, which may mount through an opening in the intraoral mount (e.g., oral device, such as an aligner, palatal expander, etc.) from within the intraoral mount, any of the apparatuses and methods described herein may include removable or replaceable sensing modules that may couple to the intraoral mount from the outside of the intraoral mount, or from both the outside and inside of the intraoral mount (or oral device). These apparatuses may couple the sensing module (e.g., thermal sensing module) to the intraoral mount mechanically, without requiring a bonding material between the sensing module and the intraoral mount/oral device. Thus, these apparatuses may be suitable for large-scale manufacturing and may allow the sensing module to be removed and/or replaced. This modular design may therefore allow the smee sensing module to be reusable for multiple times with different intraoral mounts/oral devices.

The sensing modules described herein may be configured as an electronic module that can be configured with one or more sensors to detect one or more of physical, chemical, and/or biological changes in real-time (including in temperature) that occurs in intraoral space. For example a sensing module may include one or more sensors to detect force levels, light levels, acceleration, spatial orientation, oxygen saturation, pH levels, and/or sound. The sensing module can also be configured to transmit and/or store data. The data collected can be used for compliance monitoring, health monitoring, daily activity tracking, sleep quality tracking, appliance locating, dental treatment, and oral health monitoring.

In some examples the sensing modules described herein may interlock into a specific compartment of the intraoral mount (e.g., oral device), so that the sensing module can be interchangeable with different intraoral mounts which may be configured to couple to the sensing module, and in some cases may contain the same compartment. In some cases different compartments in an intraoral mount may hold different components of the sensing module. For example, the sensing module may be interchangeable but not a power supply (e.g., battery), and the power supply may be scaled within the intraoral mount. In some examples the sensor of the sensing module may be embedded but not the data storage unit.

Any of the sensing modules described herein may be coupled to an intraoral mount (including but not limited to oral devices such as aligners, palatal expanders, etc.) by one or more mechanical locking mechanisms. A sensing module can be securely attached to an intraoral mount (e.g., intraoral appliance) and it can be configured to be detached and reused multiple times. In any of these examples the sensing module may be sealed to prevent contamination, e.g., by bodily fluids.

In some examples the sensing module housing may include a mating component, e.g., a male component and/or a female component, which is configured to secure the component to the intraoral mount (e.g., intraoral appliance). For example, the mating component may be a guide ring, protrusion, threaded projection, etc. The mating component may be integrally formed as part of the intraoral mount, e.g., by 3D printing, injection molding, etc. The mating component may be separately formed and integrated into the intraoral mount. Different materials can be used for the mating component on sensing module and/or on the intraoral mount. For instance, a metal structure can be used for a male component to provide sufficient strength for secure mounting in a small form factor to minimize overall assembly size.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or clement is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The present disclosure includes the following numbered clauses.

Clause 1. An apparatus comprising a dental appliance, the apparatus comprising: a dental appliance body forming one or more tooth-receiving cavities configured to be worn on a subject's teeth; and a sensing module coupled to the dental appliance body, the sensing module comprising: a temperature sensor; a corroborating sensor; one or more processors; and a memory coupled to the one or more processors, the memory storing instructions, that, when executed by the one or more processors, perform a method comprising: setting, during a device initialization stage, a wear-status state for the dental appliance, wherein the wear-status state is either ‘worn’ or ‘not worn’; switching the wear-status state when either or both of a change in a temperature reading from the temperature sensor exceed a temperature event threshold amount and/or a change in a corroboration sensor reading exceeds a corroboration event threshold amount; and outputting the wear-status.

Clause 2. The apparatus of clause 1, further comprising setting, during the device initialization stage, an ambient temperature value and updating the ambient temperature value when the wear-status state switched from worn to not worn.

Clause 3. The apparatus of any of clauses 1 or 2, wherein switching the wear-status state comprises switching when the temperature reading from the temperature sensor is outside of a body-temperature range and when either or both of a change in a temperature reading from the temperature sensor exceed a temperature event threshold amount and/or a change in a corroboration sensor reading exceeds a corroboration event threshold amount.

Clause 4. The apparatus of any of clauses 1-3, wherein the corroborating sensor comprises one or more of: an impedance sensor, an optical sensor, a piezoelectric sensor, a force sensor, a voltage sensor, or a pressure sensor.

Clause 5. The apparatus of any of clauses 1-4, wherein setting, during the device initialization step, the wear-status state comprises setting an ambient temperature value, further wherein switching the wear-status state comprises updating the ambient temperature value by recording a new temperature reading from the temperature sensor and setting the ambient temperature value to the new temperature reading if both the ambient temperature value and the new temperature reading are outside of a body temperature range and setting the status state to ‘not worn’, or setting the ambient temperature value to the new temperature reading and switching the state of the status state if the ambient temperature value is within the body temperature range and the new temperature reading changes more than a temperature event threshold amount from one or more immediately prior temperature readings or if the new temperature reading does not change more than the temperature event threshold amount but a change in a corroborating sensor measurement from the corroborating sensor exceeds a threshold amount, otherwise leaving the ambient temperature value and the state of the status state the same if the ambient temperature value is within the body temperature range, the new temperature does not change more than the temperature event threshold amount, and the change in the capacitance measurement does not exceed the capacitance event threshold amount.

Clause 6. The apparatus of any of clauses 1-5, wherein the corroborating sensor comprises a capacitance sensor.

Clause 7. The apparatus of clause 6, wherein the memory is further configured to determine compliance with a treatment plan on a continuous or semi-continuous manner, wherein the treatment plan specifies an amount of time that the dental appliance is required to be worn during a day, and the memory is configured to determine compliance with the treatment plan based a determination as to whether the dental appliance is being worn for specified amount of time.

Clause 8. The apparatus of any of clauses 1-7, wherein the sensing module is configured to be removably coupled to the dental appliance body.

Clause 9. The apparatus of clause 8, wherein the sensing module comprises a base having a flange that is configured to engage with an opening through the dental appliance body.

Clause 10. The apparatus of any of clauses 1-9, wherein the sensing module further comprises a force sensor configured to measure an amount of force exerted on a region of the dental appliance.

Clause 11. The apparatus of any of clauses 1-10, wherein the sensing module further comprises a pressure sensor configured to measure pressure changes within the intraoral cavity.

Clause 12. A method, the method comprising: setting, during a device initialization stage, a wear-status state for a dental appliance, wherein the wear-status state is either ‘worn’ or ‘not worn’; switching the wear-status state when either or both of a change in a temperature reading from a temperature sensor of a sensing module coupled to the dental appliance body exceeds a temperature event threshold amount and/or a change in a corroboration sensor reading from a corroboration sensor of the sensing module exceeds a corroboration event threshold amount; and outputting the wear-status.

Clause 13. The method of clause 12, further comprising setting, during the device initialization stage, an ambient temperature value and updating the ambient temperature value when the wear-status state switched from worn to not worn.

Clause 14. The method of clause 12 or 13, wherein switching the wear-status state comprises switching when the temperature reading from the temperature sensor is outside of a body-temperature range and when either or both of a change in a temperature reading from the temperature sensor exceed a temperature event threshold amount and/or a change in a corroboration sensor reading exceeds a corroboration event threshold amount.

Clause 15. The method of apparatus of any of clauses 12-14, wherein setting, during the device initialization step, the wear-status state comprises setting an ambient temperature value, further wherein switching the wear-status state comprises updating the ambient temperature value by recording a new temperature reading from the temperature sensor and setting the ambient temperature value to the new temperature reading if both the ambient temperature value and the new temperature reading are outside of a body temperature range and setting the status state to ‘not worn’, or setting the ambient temperature value to the new temperature reading and switching the state of the status state if the ambient temperature value is within the body temperature range and the new temperature reading changes more than a temperature event threshold amount from one or more immediately prior temperature readings or if the new temperature reading does not change more than the temperature event threshold amount but a change in a corroborating sensor measurement from the corroborating sensor exceeds a threshold amount, otherwise leaving the ambient temperature value and the state of the status state the same if the ambient temperature value is within the body temperature range, the new temperature does not change more than the temperature event threshold amount, and the change in the capacitance measurement does not exceed the capacitance event threshold amount.

Clause 16. The method of any of clauses 12-15, wherein the corroborating sensor comprises a capacitance sensor.

Clause 17. The method of any of clauses 12-16, further comprising removably coupling the sensor module to the dental appliance body, wherein removably coupling comprises inserting the sensing module through an opening through the dental appliance body until a flange of the sensor module engages with the opening through the dental appliance body.

Clause 18. An apparatus, the apparatus comprising: a dental appliance having a dental appliance body forming one or more tooth-receiving cavities configured to be worn on a subject's teeth; and a sensing module coupled to the dental appliance body, the sensing module comprising: one or more temperature sensors; one or more processors; and a memory coupled to the one or more processors, the memory storing instructions, that, when executed by the one or more processors, perform a method comprising: setting an ambient temperature value from the one or more temperature sensors during a device initialization stage; receiving one or more new temperature readings from one or more of the temperature sensors; updating the ambient temperature value by comparing the one or more new temperature readings to a body temperature range and comparing a change in the temperature readings to a temperature event threshold amount; and outputting the ambient temperature value.

Clause 19. A method comprising: setting an ambient temperature value during a device initialization stage based on one or more initial temperature readings from a temperature sensor of a dental appliance and setting a wear-status state for the dental appliance, wherein the wear-status state is either ‘worn’ or ‘not worn’; updating the ambient temperature value by taking a new temperature reading from the temperature sensor and setting the ambient temperature value to the new temperature reading if both the ambient temperature value and the new temperature reading are outside of a body temperature range and setting the wear-status state to ‘not worn’, or setting the ambient temperature value to the new temperature reading and switching the state of the wear-status state, if the ambient temperature value is within the body temperature range and the new temperature reading changes more than a temperature event threshold amount from one or more immediately prior temperature readings or if the new temperature reading does not change more than the temperature event threshold amount but a change in a capacitance measurement from a capacitance sensor on the dental appliance exceeds a capacitance event threshold amount, otherwise leaving the ambient temperature value and the state of the wear-status state the same if the ambient temperature value is within the body temperature range, the new temperature does not change more than the temperature event threshold amount, and the change in the capacitance measurement does not exceed the capacitance event threshold amount; and outputting the ambient temperature value.

Clause 20. The method of clause 19, wherein updating the ambient temperature value and the wear-status state comprises setting the ambient temperature value to a temperature based on the new temperature readings if both the ambient temperature value and the new temperature reading are outside of a body temperature range and setting the wear-status state to ‘not worn’, or setting the ambient temperature value to a temperature based on the new temperature reading and switching the state of the wear-status state if the ambient temperature value is within the body temperature range and the new temperature reading changes more than a temperature event threshold amount from one or more immediately prior temperature readings or if the new temperature reading does not change more than the temperature event threshold amount but a change in a capacitance measurement from a capacitance sensor on the dental appliance exceeds a capacitance event threshold amount, otherwise leaving the ambient temperature value and the state of the wear-status state the same if the ambient temperature value is within the body temperature range, the new temperature does not change more than the temperature event threshold amount, and the change in the capacitance measurement does not exceed the capacitance event threshold amount.

Clause 21. The method of any of clauses 19-20, wherein the body temperature range is between 32 and 38 degrees Celsius.

Clause 22. The method of any of clauses 19-21, wherein setting the ambient temperature value during the device initialization stage comprises setting the ambient temperature value based on all or some of the one or more initial temperature readings when at least some of the one or more initial temperature readings are outside of the body temperature range.

Clause 23. The method of clause 22, wherein setting the ambient temperature value during the device initialization stage comprises setting the ambient temperature value based on all of the one or more initial temperature readings and setting the state of the wear-status state to ‘not worn’ when all of the one or more initial temperature readings are outside of the body temperature range.

Clause 24. The method of clause 23, wherein setting the ambient temperature value during the device initialization stage comprises setting the ambient temperature value based on the one or more initial temperature readings that are outside of the body temperature range and setting the state of the wear-status state to ‘worn’ when at least one of the initial temperature readings are within the body temperature range.

Clause 25. The method of any of clauses 19-24, wherein setting the ambient temperature value during the device initialization stage comprises taking additional temperature readings from the temperature sensor, and setting the ambient temperature value based on the one or more initial temperature readings and setting the state of the wear-status state to ‘worn’ when all of the one or more initial temperature readings are within the body temperature range and either a change in the additional temperature readings exceeds the temperature event threshold amount or a change in the capacitance measurement from the capacitance sensor on the dental appliance exceeds the capacitance event threshold amount, alternatively setting the ambient temperature value based on the one or more initial temperature readings and the additional temperature readings after a predetermined device initialization time and setting the wear-status state to ‘not worn,’ when all of the one or more initial temperature readings are within the body temperature range and when neither the change in the additional temperature readings has exceeded the temperature event threshold amount nor the change in the capacitance measurement has exceeded the capacitance event threshold amount.

Clause 26. The method of any of clauses 19-25, wherein updating the ambient temperature value comprises updating for a predetermined window of time.

Clause 27. The method of any of clauses 19-26, further comprising tracking the updates to the ambient temperature value over a predetermined window of time.

Clause 28. The method of any of clauses 19-27, wherein outputting comprises outputting the ambient temperature value as a stream of time-stamped data.

Clause 29. The method of any of clauses 19-28, further comprising setting a wear-status state for the dental appliance to a value of ‘not worn’ if both the ambient temperature value and the new temperature reading are outside of the body temperature range, and switching the value of the wear-status state if the ambient temperature value is within the body temperature range and the new temperature reading changes more than the temperature event threshold amount from one or more temperature readings taken immediately prior to the new temperature reading, or if the new temperature reading does not change more than the temperature event threshold amount but the change in the capacitance measurement from the capacitance sensor on the dental appliance exceeds the capacitance event threshold amount, otherwise leaving the wear-status state value the same if the ambient temperature value is within the body temp range, the new temperature reading does not change more than the temperature event threshold amount, and the change in the capacitance measurement does not exceed the capacitance event threshold amount.

Clause 30. The method of any of clauses 19-29, wherein the temperature sensor comprises one or more of a thermistor, a resistance temperature detector (RTD), a thermocouples, and a semiconductor-based integrated sensors.

Clause 31. The method of any of clauses 19-30, wherein setting the ambient temperature value based on the one or more initial temperature readings comprises setting the ambient temperature value based on an average of two or more initial temperature readings from the temperature sensor.

Clause 32. The method of any of clauses 19-31, further comprising turning on a processor of the dental appliance to perform the device initialization stage.

Clause 33. The method of any of clauses 19-32, wherein the method is a method of determining ambient temperature from the dental appliance.

Clause 34. An apparatus, the apparatus comprising: a dental appliance having a dental appliance body forming one or more tooth-receiving cavities configured to be worn on a subject's teeth; and a sensing module coupled to the dental appliance body, the sensing module comprising: one or more temperature sensors; one or more proximity sensors; one or more processors; and a memory coupled to the one or more processors, the memory storing instructions, that, when executed by the one or more processors, perform a method comprising: setting an ambient temperature value during a device initialization stage based on one or more initial temperature readings from the one or more temperature sensors; updating the ambient temperature value by taking a new temperature reading from the one or more temperature sensors and setting the ambient temperature value to the new temperature reading if both the ambient temperature value and the new temperature reading are outside of a body temperature range, or setting the ambient temperature value to the new temperature reading if the ambient temperature value is within the body temperature range and the new temperature reading changes more than a temperature event threshold amount from one or more immediately prior temperature readings or if the new temperature reading does not change more than the temperature event threshold amount but a change in a proximity measurement from the one or more proximity sensor exceeds a proximity event threshold amount, otherwise leaving the ambient temperature value the same if the ambient temperature value is within the body temperature range, the new temperature does not change more than the temperature event threshold amount, and the change in the proximity measurement does not exceed the proximity event threshold amount; and outputting the ambient temperature value.

Clause 35. The apparatus of clause 34, wherein the sensing module comprises one or more capacitive sensors, further wherein the updating comprises comparing a change in a proximity measurement from the one or more proximity sensors to a proximity activity threshold amount when the temperature reading is within the body temperature range and the temperature reading changes more than the temperature activity threshold amount.

Clause 36. The apparatus of clause 34, wherein updating the ambient temperature value comprises: setting the ambient temperature value to the new temperature reading if both the ambient temperature value and the new temperature reading are outside of the body temperature range, or setting the ambient temperature value to the new temperature reading if the ambient temperature value is within the body temperature range and the new temperature reading changes more than a temperature event threshold amount from one or more immediately prior temperature readings or if the new temperature reading does not change more than the temperature event threshold amount but a change in the proximity measurement exceeds the proximity event threshold amount, otherwise leaving the ambient temperature value the same.

Clause 37. The apparatus of any of clauses 34-36, wherein the proximity sensor comprises one or more of: a capacitance sensor or an optical sensor.

Clause 38. The apparatus of any of clauses 34-37, wherein the body temperature range is between 32 and 38 degrees Celsius.

Clause 39. The apparatus of any of clauses 34-38, wherein setting the ambient temperature value during the device initialization stage comprises setting the ambient temperature value based on all or some of one or more initial temperature readings when at least some of the one or more initial temperature readings are outside of the body temperature range.

Clause 40. The apparatus of clause 39 wherein setting the ambient temperature value during the device initialization stage comprises setting the ambient temperature value based on all of the one or more initial temperature readings when all of the one or more initial temperature readings are outside of the body temperature range.

Clause 41. The apparatus of clause 39, wherein setting the ambient temperature value during the device initialization stage comprises setting the ambient temperature value based on the one or more initial temperature readings that are outside of the body temperature range when at least one of the initial temperature readings are within the body temperature range.

Clause 42. The apparatus of any of clauses 34-41, wherein setting the ambient temperature value during the device initialization stage comprises taking additional temperature readings from the one or more temperature sensors, and setting the ambient temperature value based on the one or more initial temperature readings when all of the one or more initial temperature readings are within the body temperature range and either a change in the additional temperature readings exceeds the temperature event threshold amount or a change in the proximity measurement from the one or more proximity sensors on the dental appliance exceeds the proximity event threshold amount, alternatively setting the ambient temperature value based on the one or more initial temperature readings and the additional temperature readings after a predetermined device initialization time when all of the one or more initial temperature readings are within the body temperature range and when neither the change in the additional temperature readings has exceeded the temperature event threshold amount nor the change in the proximity measurement has exceeded the proximity event threshold amount.

Clause 43. The apparatus of any of clauses 34-42, wherein updating the ambient temperature value comprises updating for a predetermined window of time.

Clause 44. The apparatus of any of clauses 34-43, further comprising tracking the updates to the ambient temperature value over a predetermined window of time.

Clause 45. The apparatus of any of clauses 34-44, wherein outputting comprises outputting the ambient temperature value as a stream of time-stamped data.

Clause 46. The apparatus of any of clauses 34-45, wherein outputting comprises outputting the ambient temperature value and the new temperature readings as a stream of time-stamped data.

Clause 47. The apparatus of any of clauses 34-46, wherein setting comprises setting the ambient temperature value to the new temperature reading if both the ambient temperature value and the new temperature reading are outside of the body temperature range.

Clause 48. The apparatus of any of clauses 34-47, wherein setting comprises setting the ambient temperature value to the new temperature reading if the ambient temperature value is within the body temperature range and the new temperature reading changes more than a temperature event threshold amount from one or more immediately prior temperature readings or if the new temperature reading does not change more than the temperature event threshold amount but a change in the proximity measurement exceeds the proximity event threshold amount.

Clause 49. The apparatus of any of clauses 34-48, wherein setting the ambient temperature value during the device initialization stage further comprises setting a wear-status state for the dental appliance to ‘not worn’ if either all of one or more temperature readings during the device initialization stage are outside of the body temperature range, or if all of the one or more temperature readings during the device initialization stage are within the body temperature range and neither a change in an additional temperature reading has exceeded the temperature event threshold amount nor the change in the proximity measurement has exceeded the proximity event threshold amount, otherwise setting the wear-status state to ‘worn’.

Clause 50. The apparatus of any of clauses 34-49, further comprising setting a wear-status state for the dental appliance to a value of ‘not worn’ if both the ambient temperature value and the new temperature reading are outside of the body temperature range, and switching the value of the wear-status state if the ambient temperature value is within the body temperature range and the new temperature reading changes more than the temperature event threshold amount from one or more temperature readings taken immediately prior to the new temperature reading, or if the new temperature reading does not change more than the temperature event threshold amount but the change in the proximity measurement from the one or more proximity sensors on the dental appliance exceeds the proximity event threshold amount, otherwise leaving a value of the wear-status state the same if the ambient temperature value is within the body temp range, the new temperature reading does not change more than the temperature event threshold amount, and the change in the proximity measurement does not exceed the proximity event threshold amount.

Clause 51. The apparatus of any of clauses 34-50, wherein the one or more temperature sensors comprises one or more of a thermistor, a resistance temperature detector (RTD), a thermocouples, and a semiconductor-based integrated sensors.

Clause 52. The apparatus of any of clauses 34-51, wherein setting the ambient temperature value based on the one or more initial temperature readings comprises setting the ambient temperature value based on an average of two or more initial temperature readings from the one or more temperature sensors.

Clause 53. An apparatus, the apparatus comprising: a dental appliance body forming one or more tooth-receiving cavities configured to be worn on a subject's teeth; and a sensing module coupled to the dental appliance body, the sensing module comprising: one or more temperature sensors; one or more capacitance sensors; one or more processors; and a memory coupled to the one or more processors, the memory storing instructions, that, when executed by the one or more processors, perform a method comprising: setting, during a device initialization stage, an ambient temperature value from the temperature sensor and setting a wear-status state for the dental appliance, wherein the wear-status state is either ‘worn’ or ‘not worn’; updating, on an ongoing basis, the ambient temperature value and the wear-status state using the one or more temperature sensors by comparing one or more new temperature readings from the one or more temperature sensors to a body temperature range and to a temperature event threshold amount, and by comparing a capacitance measurement from the one or more capacitance sensors to a capacitance event threshold amount when the temperature reading is within the body temperature range and the temperature reading changes more than the temperature event threshold amount; and outputting the ambient temperature value and/or the wear-status state.

Clause 54. An apparatus, the apparatus comprising: a dental appliance body forming one or more tooth-receiving cavities configured to be worn on a subject's teeth; and a sensing module coupled to the dental appliance body, the sensing module comprising: one or more temperature sensors; one or more capacitance sensors; one or more processors; and a memory coupled to the one or more processors, the memory storing instructions, that, when executed by the one or more processors, perform a method comprising: setting an ambient temperature value during a device initialization stage based on one or more initial temperature readings from the one or more temperature sensors and setting a wear-status state for the dental appliance, wherein the wear-status state is either ‘worn’ or ‘not worn’; updating the ambient temperature value by recording a new temperature reading from the one or more temperature sensors and setting the ambient temperature value to the new temperature reading if both the ambient temperature value and the new temperature reading are outside of a body temperature range and setting the wear-status state to ‘not worn’, or setting the ambient temperature value to the new temperature reading and switching the state of the wear-status state, if the ambient temperature value is within the body temperature range and the new temperature reading changes more than a temperature event threshold amount from one or more immediately prior temperature readings or if the new temperature reading does not change more than the temperature event threshold amount but a change in a capacitance measurement from the one or more capacitance sensors exceeds a capacitance event threshold amount, otherwise leaving the ambient temperature value and the state of the wear-status state the same if the ambient temperature value is within the body temperature range, the new temperature does not change more than the temperature event threshold amount, and the change in the capacitance measurement does not exceed the capacitance event threshold amount; and outputting the ambient temperature value and/or the wear-status state.

Clause 55. The apparatus of clause 53, wherein updating the ambient temperature value and the wear-status state comprises setting the ambient temperature value to a temperature based on the new temperature readings if both the ambient temperature value and the new temperature reading are outside of a body temperature range and setting the wear-status state to ‘not worn’, or setting the ambient temperature value to a temperature based on the new temperature reading and switching the state of the wear-status state if the ambient temperature value is within the body temperature range and the new temperature reading changes more than a temperature event threshold amount from one or more immediately prior temperature readings or if the new temperature reading does not change more than the temperature event threshold amount but a change in a capacitance measurement from the one or more capacitance sensors exceeds a capacitance event threshold amount, otherwise leaving the ambient temperature value and the state of the wear-status state the same if the ambient temperature value is within the body temperature range, the new temperature does not change more than the temperature event threshold amount, and the change in the capacitance measurement does not exceed the capacitance event threshold amount.

Clause 56. The apparatus of any of clauses 54-55, wherein the body temperature range is between 32 and 38 degrees Celsius.

Clause 57. The apparatus of any of clauses 54-56, wherein setting the ambient temperature value during the device initialization stage comprises setting the ambient temperature value based on all or some of one or more initial temperature readings when at least some of the one or more initial temperature readings are outside of the body temperature range.

Clause 58. The apparatus of clause 57, wherein setting the ambient temperature value during the device initialization stage comprises setting the ambient temperature value based on all of the one or more initial temperature readings when all of the one or more initial temperature readings are outside of the body temperature range.

Clause 59. The apparatus of clause 57, wherein setting the ambient temperature value during the device initialization stage comprises setting the ambient temperature value based on the one or more initial temperature readings that are outside of the body temperature range when at least one of the initial temperature readings are within the body temperature range.

Clause 60. The apparatus of any of clauses 54-59, wherein setting the ambient temperature value during the device initialization stage comprises taking additional temperature readings from the temperature sensor, and setting the ambient temperature value based on the one or more initial temperature readings when all of the one or more initial temperature readings are within the body temperature range and either a change in the additional temperature readings exceeds the temperature event threshold amount or a change in the capacitance measurement from the one or more capacitance sensors exceeds the capacitance event threshold amount, alternatively setting the ambient temperature value based on the one or more initial temperature readings and the additional temperature readings after a predetermined device initialization time when all of the one or more initial temperature readings are within the body temperature range and when neither the change in the additional temperature readings has exceeded the temperature event threshold amount nor the change in the capacitance measurement has exceeded the capacitance event threshold amount.

Clause 61. The apparatus of any of clauses 54-60, wherein updating the ambient temperature value comprises updating for a predetermined window of time.

Clause 62. The apparatus of any of clauses 54-61, further comprising tracking the updates to the ambient temperature value over a predetermined window of time.

Clause 63. The apparatus of any of clauses 54-62, wherein outputting comprises outputting the ambient temperature value and/or the wear-status state as a stream of time-stamped data.

Clause 64. The apparatus of any of clauses 54-63, wherein outputting comprises outputting the ambient temperature value and the new temperature readings and/or the wear-status state as a stream of time-stamped data.

Clause 65. The apparatus of any of clauses 54-64, wherein setting the ambient temperature value during the device initialization stage further comprises setting a wear-status state for the dental appliance to ‘not worn’ if either all of one or more temperature readings during the device initialization stage are outside of the body temperature range, or if all of the one or more temperature readings during the device initialization stage are within the body temperature range and neither a change in an additional temperature reading has exceeded the temperature event threshold amount nor the change in the capacitance measurement has exceeded the capacitance event threshold amount, otherwise setting the wear-status state to ‘worn’.

Clause 66. The apparatus of any of clauses 54-65, further comprising setting a wear-status state for the dental appliance to a value of ‘not worn’ if both the ambient temperature value and the new temperature reading are outside of the body temperature range, and switching the value of the wear-status state if the ambient temperature value is within the body temperature range and the new temperature reading changes more than the temperature event threshold amount from one or more temperature readings taken immediately prior to the new temperature reading, or if the new temperature reading does not change more than the temperature event threshold amount but the change in the capacitance measurement from the one or more capacitance sensors exceeds the capacitance event threshold amount, otherwise leaving a value of the wear-status state the same if the ambient temperature value is within the body temp range, the new temperature reading does not change more than the temperature event threshold amount, and the change in the capacitance measurement does not exceed the capacitance event threshold amount.

Clause 67. The apparatus of any of clauses 54-66, wherein the one or more temperature sensors comprises one or more of a thermistor, a resistance temperature detector (RTD), a thermocouples, and a semiconductor-based integrated sensors.

Clause 68. The apparatus of any of clauses 54-67, wherein setting the ambient temperature value based on the one or more initial temperature readings comprises setting the ambient temperature value based on an average of two or more initial temperature readings from the one or more temperature sensors.

Clause 69. A method, the method comprising: setting an ambient temperature value from a temperature sensor on a dental appliance during a device initialization stage; updating, on an ongoing basis, the ambient temperature value using the temperature sensor on the dental appliance by comparing one or more new temperature readings from the temperature sensor to a body temperature range and to a temperature event threshold amount, and by comparing a capacitance measurement from a capacitance sensor on the dental appliance to a capacitance event threshold amount when the temperature reading is within the body temperature range and the temperature reading changes more than the temperature event threshold amount; and outputting the ambient temperature value.

Clause 70. A method comprising: setting an ambient temperature value during a device initialization stage based on one or more initial temperature readings from a temperature sensor of a dental appliance; updating the ambient temperature value by taking a new temperature reading from the temperature sensor and setting the ambient temperature value to the new temperature reading if both the ambient temperature value and the new temperature reading are outside of a body temperature range, or setting the ambient temperature value to the new temperature reading if the ambient temperature value is within the body temperature range and the new temperature reading changes more than a temperature event threshold amount from one or more immediately prior temperature readings or if the new temperature reading does not change more than the temperature event threshold amount but a change in a capacitance measurement from a capacitance sensor on the dental appliance exceeds a capacitance event threshold amount, otherwise leaving the ambient temperature value the same if the ambient temperature value is within the body temperature range, the new temperature does not change more than the temperature event threshold amount, and the change in the capacitance measurement does not exceed the capacitance event threshold amount; and outputting the ambient temperature value.

Clause 71. The method of clause 69, wherein updating the ambient temperature value comprises setting the ambient temperature value to the new temperature reading if both the ambient temperature value and the new temperature reading are outside of the body temperature range, or setting the ambient temperature value to the new temperature reading if the ambient temperature value is within the body temperature range and the new temperature reading changes more than a temperature event threshold amount from one or more immediately prior temperature readings or if the new temperature reading does not change more than the temperature event threshold amount but a change in the capacitance measurement exceeds the capacitance event threshold amount, otherwise leaving the ambient temperature value the same.

Clause 72. The method of any of clauses 70-71, wherein the body temperature range is between 32 and 38 degrees Celsius.

Clause 73. The method of any of clauses 70-72, wherein setting the ambient temperature value during the device initialization stage comprises setting the ambient temperature value based on all or some of one or more initial temperature readings when at least some of the one or more initial temperature readings are outside of the body temperature range.

Clause 74. The method of clause 73, wherein setting the ambient temperature value during the device initialization stage comprises setting the ambient temperature value based on all of the one or more initial temperature readings when all of the one or more initial temperature readings are outside of the body temperature range.

Clause 75. The method of clause 73, wherein setting the ambient temperature value during the device initialization stage comprises setting the ambient temperature value based on the one or more initial temperature readings that are outside of the body temperature range when at least one of the initial temperature readings are within the body temperature range.

Clause 76. The method of any of clauses 70-75, wherein setting the ambient temperature value during the device initialization stage comprises taking additional temperature readings from the temperature sensor, and setting the ambient temperature value based on the one or more initial temperature readings when all of the one or more initial temperature readings are within the body temperature range and either a change in the additional temperature readings exceeds the temperature event threshold amount or a change in the capacitance measurement from the capacitance sensor on the dental appliance exceeds the capacitance event threshold amount, alternatively setting the ambient temperature value based on the one or more initial temperature readings and the additional temperature readings after a predetermined device initialization time when all of the one or more initial temperature readings are within the body temperature range and when neither the change in the additional temperature readings has exceeded the temperature event threshold amount nor the change in the capacitance measurement has exceeded the capacitance event threshold amount.

Clause 77. The method of any of clauses 70-76, wherein updating the ambient temperature value comprises updating for a predetermined window of time.

Clause 78. The method of any of clauses 70-77, further comprising tracking the updates to the ambient temperature value over a predetermined window of time.

Clause 79. The method of any of clauses 70-78, wherein outputting comprises outputting the ambient temperature value as a stream of time-stamped data.

Clause 80. The method of any of clauses 70-79, wherein outputting comprises outputting the ambient temperature value and the new temperature readings as a stream of time-stamped data.

Clause 81. The method of any of clauses 70-80, wherein setting the ambient temperature value during the device initialization stage further comprises setting a wear-status state for the dental appliance to ‘not worn’ if either all of one or more temperature readings during the device initialization stage are outside of the body temperature range, or if all of the one or more temperature readings during the device initialization stage are within the body temperature range and neither a change in an additional temperature reading has exceeded the temperature event threshold amount nor the change in the capacitance measurement has exceeded the capacitance event threshold amount, otherwise setting the wear-status state to ‘worn’.

Clause 82. The method of any of clauses 70-81, further comprising setting a wear-status state for the dental appliance to a value of ‘not worn’ if both the ambient temperature value and the new temperature reading are outside of the body temperature range, and switching the value of the wear-status state if the ambient temperature value is within the body temperature range and the new temperature reading changes more than the temperature event threshold amount from one or more temperature readings taken immediately prior to the new temperature reading, or if the new temperature reading does not change more than the temperature event threshold amount but the change in the capacitance measurement from the capacitance sensor on the dental appliance exceeds the capacitance event threshold amount, otherwise leaving a value of the wear-status state the same if the ambient temperature value is within the body temp range, the new temperature reading does not change more than the temperature event threshold amount, and the change in the capacitance measurement does not exceed the capacitance event threshold amount.

Clause 83. The method of any of clauses 70-82, wherein the temperature sensor comprises one or more of a thermistor, a resistance temperature detector (RTD), a thermocouples, and a semiconductor-based integrated sensors.

Clause 84. The method of any of clauses 70-83, wherein the method is a method of determining ambient temperature from the dental appliance.

Clause 85. The method of any of clauses 70-84, further comprising turning on a processor of the dental appliance to perform the device initialization stage.

Clause 86. A method comprising: setting, during a device initialization stage, an ambient temperature value from the temperature sensor and setting a wear-status state for the dental appliance, wherein the wear-status state is either ‘worn’ or ‘not worn’; updating, on an ongoing basis, the ambient temperature value and the wear-status state using the temperature sensor by comparing one or more new temperature readings from the temperature sensor to a body temperature range and to a temperature event threshold amount, and by comparing a capacitance measurement from the capacitance sensor to a capacitance event threshold amount when the temperature reading is within the body temperature range and the temperature reading changes more than the temperature event threshold amount; and outputting the ambient temperature value.

Clause 87. A method of determining ambient temperature from a dental appliance, the method comprising: determining if the dental appliance is worn or not worn in a mouth based on a temperature measured from a temperature sensor on the dental appliance, a change in the temperature measured temperature from the temperature sensor over time; setting an ambient temperature value to the temperature measured from the temperature sensor on dental appliance if the dental appliance is determined to be not worn; and outputting the ambient temperature value.

Clause 88. The method of clause 87, wherein determining if the dental appliance is worn or not worn in a mouth is based on the temperature measured from a temperature sensor on the dental appliance, the change in the temperature measured temperature from the temperature sensor over time and a signal from a proximity sensor over time.

Clause 89. The method of clause 87, wherein determining if the dental appliance is worn or not worn in a mouth comprises comparing a temperature measured from a temperature sensor to a body temperature range, comparing the change in the temperature measured from the temperature sensor over time to a temperature activity threshold amount, and by comparing a change in the signal from the proximity sensor over time to a proximity activity threshold amount when the temperature measured from a temperature sensor is within the body temperature range and the change in the temperature measured from a temperature sensor changes more than the temperature activity threshold amount.

Clause 90. The method of any of clauses 87-89, wherein the proximity sensor comprises a capacitance sensor.

Clause 91. An apparatus comprising a dental appliance, the apparatus comprising: a dental appliance body forming one or more tooth-receiving cavities configured to be worn on a subject's teeth; and a sensing module coupled to the dental appliance body, the sensing module comprising: a temperature sensor; a corroborating sensor; one or more processors; and a memory coupled to the one or more processors, the memory storing instructions, that, when executed by the one or more processors, perform a method comprising: determining if the dental appliance is worn or not worn in a mouth based on a temperature measured from a temperature sensor on the dental appliance, a change in the temperature measured temperature from the temperature sensor over time; setting an ambient temperature value to the temperature measured from the temperature sensor on dental appliance if the dental appliance is determined to be not worn; and outputting the ambient temperature value.

What is claimed is:

1. An apparatus comprising a dental appliance, the apparatus comprising:
a dental appliance body forming one or more tooth-receiving cavities configured to be worn on a subject's teeth; and
a sensing module coupled to the dental appliance body, the sensing module comprising:
a temperature sensor;
a corroborating sensor;
one or more processors; and
a memory coupled to the one or more processors, the memory storing instructions, that, when executed by the one or more processors, perform a method comprising:
setting, during a device initialization stage, a wear-status state for the dental appliance, wherein the wear-status state is either ‘worn’ or ‘not worn’;
switching the wear-status state when either or both of a change in a temperature reading from the temperature sensor exceed a temperature event threshold amount and/or a change in a corroboration sensor reading exceeds a corroboration event threshold amount; and
outputting the wear-status.

2. The apparatus of claim 1, further comprising setting, during the device initialization stage, an ambient temperature value and updating the ambient temperature value when the wear-status state switched from worn to not worn.

3. The apparatus of claim 1, wherein switching the wear-status state comprises switching when the temperature reading from the temperature sensor is outside of a body-temperature range and when either or both of a change in a temperature reading from the temperature sensor exceed a temperature event threshold amount and/or a change in a corroboration sensor reading exceeds a corroboration event threshold amount.

4. The apparatus of claim 1, wherein the corroborating sensor comprises one or more of: an impedance sensor, an optical sensor, a piezoelectric sensor, a force sensor, a voltage sensor, or a pressure sensor.

5. The apparatus of claim 1, wherein setting, during the device initialization step, the wear-status state comprises setting an ambient temperature value, further wherein switching the wear-status state comprises updating the ambient temperature value by recording a new temperature reading from the temperature sensor and setting the ambient temperature value to the new temperature reading if both the ambient temperature value and the new temperature reading are outside of a body temperature range and setting the status state to ‘not worn’, or setting the ambient temperature value to the new temperature reading and switching the state of the status state if the ambient temperature value is within the body temperature range and the new temperature reading changes more than a temperature event threshold amount from one or more immediately prior temperature readings or if the new temperature reading does not change more than the temperature event threshold amount but a change in a corroborating sensor measurement from the corroborating sensor exceeds a threshold amount, otherwise leaving the ambient temperature value and the state of the status state the same if the ambient temperature value is within the body temperature range, the new temperature does not change more than the temperature event threshold amount, and the change in the capacitance measurement does not exceed the capacitance event threshold amount.

6. The apparatus of claim 1, wherein the corroborating sensor comprises a capacitance sensor.

7. The apparatus of claim 6, wherein the memory is further configured to determine compliance with a treatment plan on a continuous or semi-continuous manner, wherein the treatment plan specifies an amount of time that the dental appliance is required to be worn during a day, and the memory is configured to determine compliance with the treatment plan based a determination as to whether the dental appliance is being worn for specified amount of time.

8. The apparatus of claim 1, wherein the sensing module is configured to be removably coupled to the dental appliance body.

9. The apparatus of claim 8, wherein the sensing module comprises a base having a flange that is configured to engage with an opening through the dental appliance body.

10. The apparatus of claim 1, wherein the sensing module further comprises a force sensor configured to measure an amount of force exerted on a region of the dental appliance.

11. The apparatus of claim 1, wherein the sensing module further comprises a pressure sensor configured to measure pressure changes within the intraoral cavity.

12. A method, the method comprising:
setting, during a device initialization stage, a wear-status state for a dental appliance, wherein the wear-status state is either ‘worn’ or ‘not worn’;
switching the wear-status state when either or both of a change in a temperature reading from a temperature sensor of a sensing module coupled to the dental appliance body exceeds a temperature event threshold amount and/or a change in a corroboration sensor reading from a corroboration sensor of the sensing module exceeds a corroboration event threshold amount; and
outputting the wear-status.

13. The method of claim 12, further comprising setting, during the device initialization stage, an ambient temperature value and updating the ambient temperature value when the wear-status state switched from worn to not worn.

14. The method of claim 12, wherein switching the wear-status state comprises switching when the temperature reading from the temperature sensor is outside of a body-temperature range and when either or both of a change in a temperature reading from the temperature sensor exceed a temperature event threshold amount and/or a change in a corroboration sensor reading exceeds a corroboration event threshold amount.

15. The method of claim 12, wherein setting, during the device initialization step, the wear-status state comprises setting an ambient temperature value, further wherein switching the wear-status state comprises updating the ambient temperature value by recording a new temperature reading from the temperature sensor and setting the ambient temperature value to the new temperature reading if both the ambient temperature value and the new temperature reading are outside of a body temperature range and setting the status state to ‘not worn’, or setting the ambient temperature value to the new temperature reading and switching the state of the status state if the ambient temperature value is within the body temperature range and the new temperature reading changes more than a temperature event threshold amount from one or more immediately prior temperature readings or if the new temperature reading does not change more than the temperature event threshold amount but a change in a corroborating sensor measurement from the corroborating sensor exceeds a threshold amount, otherwise leaving the ambient temperature value and the state of the status state the same if the ambient temperature value is within the body temperature range, the new temperature does not change more than the temperature event threshold amount, and the change in the capacitance measurement does not exceed the capacitance event threshold amount.

16. The method of claim 12, wherein the corroborating sensor comprises a capacitance sensor.

17. The method of claim 12, further comprising removably coupling the sensor module to the dental appliance body, wherein removably coupling comprises inserting the sensing module through an opening through the dental appliance body until a flange of the sensor module engages with the opening through the dental appliance body.

18. An apparatus, the apparatus comprising:
a dental appliance having a dental appliance body forming one or more tooth-receiving cavities configured to be worn on a subject's teeth; and
a sensing module coupled to the dental appliance body, the sensing module comprising:
one or more temperature sensors;
one or more processors; and
a memory coupled to the one or more processors, the memory storing instructions, that, when executed by the one or more processors, perform a method comprising:
setting an ambient temperature value from the one or more temperature sensors during a device initialization stage;
receiving one or more new temperature readings from one or more of the temperature sensors;
updating the ambient temperature value by comparing the one or more new temperature readings to a body temperature range and comparing a change in the temperature readings to a temperature event threshold amount; and
outputting the ambient temperature value.

19. A method comprising:
setting an ambient temperature value during a device initialization stage based on one or more initial temperature readings from a temperature sensor of a dental appliance and setting a wear-status state for the dental appliance, wherein the wear-status state is either ‘worn’ or ‘not worn’;
updating the ambient temperature value by taking a new temperature reading from the temperature sensor and setting the ambient temperature value to the new temperature reading if both the ambient temperature value and the new temperature reading are outside of a body temperature range and setting the wear-status state to ‘not worn’, or setting the ambient temperature value to the new temperature reading and switching the state of the wear-status state, if the ambient temperature value is within the body temperature range and the new temperature reading changes more than a temperature event threshold amount from one or more immediately prior temperature readings or if the new temperature reading does not change more than the temperature event threshold amount but a change in a capacitance measurement from a capacitance sensor on the dental appliance exceeds a capacitance event threshold amount, otherwise leaving the ambient temperature value and the state of the wear-status state the same if the ambient temperature value is within the body temperature range, the new temperature does not change more than the temperature event threshold amount, and the change in the capacitance measurement does not exceed the capacitance event threshold amount; and
outputting the ambient temperature value.
